(19) United States
(12) Patent Application Publication
(10) Pub. No.: US 2024/0344065 A1
(43) Pub. Date: Oct. 17, 2024

(54) METHOD

(71) Applicant: Oxford University Innovation Limited, Oxford (GB)

(72) Inventors: Charles Thomas ROBERTS, Oxford (Oxfordshire) (GB); Matthew WOOD, Oxford (Oxfordshire) (GB)

(21) Appl. No.: 18/294,324

(22) PCT Filed: Aug. 4, 2022
(86) PCT No.: PCT/GB2022/052052

§ 371 Date: Feb. 1, 2024

(30) Foreign Application Priority Data
Aug. 4, 2021 (GB) 2111250.3

(51) Int. Cl. C12N 15/113; C12N 15/86

(52) U.S. Cl. CPC C12N 15/113; C12N 15/86; C12N 2310/11; C12N 2310/322; C12N 2310/351; C12N 2320/33; C12N 2740/15043; C12N 2750/14143

(57) ABSTRACT

The invention relates to modulating the expression or activity of a gene by modulating the presence of a regulatory element in the corresponding RNA transcript using a compound targeted to a splicing signal in the RNA transcript to induce splice modulation of one or more exons comprising the regulatory element.

### FIELD OF INVENTION

The invention relates to compounds for modulating gene expression or activity, and methods and uses thereof.

### BACKGROUND TO THE INVENTION

Techniques for modulating the expression of endogenous genes are known in the art. For example, the modulation of gene expression can be mediated at the level of transcription, such as by DNA-binding agents, small molecules or synthetic oligonucleotides. Gene expression may also be modulated post-transcriptionally, e.g. through RNA interference.

Targeted gene silencing technologies are relatively well-developed, including antisense technology, small interfering RNA (siRNA) technology, and CRISPR interference technology. However, there are relatively few technologies which can induce targeted gene upregulation.

It is an object of the invention to identify further and improved compounds and methods for modulating the expression or activity of specific genes.

### SUMMARY OF THE INVENTION

The inventors identified a new way of modulating the expression or activity of specific genes. In particular, the inventors discovered that many regulatory elements that have a significant impact on gene expression or activity, such as upstream open reading frames (uORFs), are located in the exons of a RNA transcript. As such, splice modulation can be utilised to alter the presence or absence of specific exons, and hence the regulatory elements contained therein.

The removal of a regulatory sequence as a consequence of induced splice modulation would result in reversing the effect of that regulatory element. For example, splice modulation to remove an exon that contains a negative regulatory element (e.g. uORF) would generate a RNA transcript which lacks the negative regulatory element, and thereby induces up-regulation of the protein that the regulatory element would have naturally regulated. Alternatively, splice modulation to remove an exon that contains a positive regulatory element (e.g. a transcript stabilising motif) would generate a RNA transcript which lacks the positive regulatory element, and thereby induces down-regulation of the protein that the regulatory element would have naturally regulated. As a further example, splice modulation to remove an exon that contains a sub-cellular localisation signal would alter the sub-cellular localisation of that transcript. As another example, splice modulation to remove an exon that contains regulatory elements which interact with other RNAs or proteins in a non-coding RNA transcript would lead to loss of specific interactions, and thereby modulate the activity of the non-coding RNA transcript.

Conversely, the inclusion of a regulatory sequence as a consequence of induced splice modulation would have the effect of promoting outcomes dependent on the nature of the regulatory element present in the newly included exon. For example, the inclusion of an alternatively spliced or cryptic exon containing a negative regulatory element (e.g. uORF) would generate a RNA transcript which additionally contains the negative regulatory element, and thereby induces down-regulation of the protein that the regulatory element would have naturally regulated. Alternatively, the inclusion of an alternatively spliced or cryptic exon containing a positive regulatory element (e.g. a transcript stabilising motif) would generate a RNA transcript which additionally contains the positive regulatory element, and thereby induces up-regulation of the protein that the regulatory element would have naturally regulated. As a further example, splice modulation to include an exon that contains a sub-cellular localisation signal would alter the sub-cellular localisation of that transcript.

Accordingly, the invention provides a method for modulating the presence of a regulatory element in a RNA transcript, comprising delivering to a cell a compound targeted to a splicing signal in the RNA transcript to induce splice modulation of one or more exons comprising a regulatory element.

The invention also provides a method of modulating the expression or activity of a gene, comprising modulating the presence of a regulatory element in the RNA transcript encoded by the gene according to the method of the invention.

The invention also provides a method of increasing, decreasing or restoring protein expression, comprising modulating the presence of a regulatory element in a RNA transcript according to the method of the invention.

The invention also provides an oligonucleotide targeted to a splicing signal in a RNA transcript for inducing alternative splicing, such that one or more exons comprising a regulatory element are skipped and/or retained.

The invention also provides a conjugated oligonucleotide comprising two or more oligonucleotide of the invention.

The invention also provides a polynucleotide or a vector encoding the oligonucleotide or conjugated oligonucleotide of the invention, optionally wherein the vector is AAV or lentivirus.

The invention also provides a delivery vehicle comprising an oligonucleotide or conjugated oligonucleotide of the invention.

The invention also provides a modified RNA transcript comprising the absence or inclusion of one or more exons comprising a regulatory element compared to the unmodified RNA transcript.

The invention also provides a composition comprising two or more oligonucleotides according to the invention, optionally wherein the oligonucleotides are conjugated.

The invention also provides a pharmaceutical composition comprising the oligonucleotide, the conjugated oligonucleotide, the polynucleotide or vector, the delivery vehicle, or the composition of the invention, and a pharmaceutically acceptable carrier.

The invention also provides an oligonucleotide, a conjugated oligonucleotide, a polynucleotide or vector, a delivery vehicle, a composition or a pharmaceutical composition of the invention for use in a method of therapy practised on the human or animal body.

The invention also provides an oligonucleotide, a conjugated oligonucleotide, a polynucleotide or vector, a delivery vehicle, a composition or a pharmaceutical composition according to the invention for use in a method of treating or preventing a disease or condition in a subject by modulating the expression of a gene, comprising administering to the subject a therapeutically effective amount of the oligonucleotide, the polynucleotide, the delivery vehicle, the composition or the pharmaceutical composition.

The invention also provides the use of an oligonucleotide, a conjugated oligonucleotide, a polynucleotide or vector, a delivery vehicle, a composition or a pharmaceutical composition according to the invention in the manufacture of a medicament for the treatment or prevention of a disease or condition in a subject by modulating the expression or activity of a gene.

The invention also provides the use of an oligonucleotide, a conjugated oligonucleotide, a polynucleotide or vector, a delivery vehicle, a composition or a pharmaceutical composition according to the invention for the treatment or prevention of a disease or condition in a subject by modulating the expression or activity of a gene.

The invention also provides a method of treating or preventing a disease or condition in a subject by modulating the expression or activity of a gene, comprising administering to the subject a therapeutically effective amount of the oligonucleotide or conjugated oligonucleotide, the polynucleotide or vector, the delivery vehicle, the composition or the pharmaceutical composition of the invention.

### BRIEF DESCRIPTION OF THE FIGURES

FIG. 1A shows the proportion of human and mouse transcripts containing predicted uORFs. FIG. 1B shows the number of uORFs per transcript. FIG. 1C shows the distribution of uORF lengths. FIG. 1D shows the proportion of uORFs which overlap with the pORF. FIG. 1E shows the distribution of distances between the transcription start site and uORF. FIG. 1F shows the distribution of distances between the uORF and the pORF. FIG. 1G shows the distribution of pORF and uORF stop codon usage. FIG. 1H shows the proportion of uORFs and pORFs with weak or strong Kozak contexts. FIG. 1I shows logo plots for the Kozak context at uORFs and pORFs. FIG. 1J shows the distribution of phastCons scores in uORFs relative to other genomic features.

FIG. 1K shows a predicted uORF at the HTT gene mapped on to the genome browser with additional riboseq and RNA-seq tracks.

FIG. 2A shows luciferase validation data for predicted uORFs for FOXL2, HOXA11, JUN, KDR, RNASEH1, SMO and SRY. Each 5′ UTR was cloned upstream of a Renilla reporter gene and a mutant construct generated in which the uORF ATG was changed to TTG, thereby inactivating the uORF. Activation of luciferase expression is indicative of de-repression uORF-mediated translational repression. FIG. 2B shows transcript level data for the constructs in FIG. 2A. FIG. 2C shows an independent verification of FIG. 2A that also includes MAP2K2. FIG. 2D shows luciferase reporter data for BDNF (2 isoforms), C9orf72, GATA2 (3 isoforms), GDNF, HTT and SCN1A. Together these plots validate the existence of multiple uORFs. FIG. 2E shows a cumulative distribution function plot for proteomics data (ubiquitously-expressed proteins only) taken from 29 healthy human tissues (1) whereby transcripts were classified as uORF-containing or non-uORF-containing. The distribution of protein expression values is significantly lower for uORF-containing transcripts. FIG. 2F shows a cumulative distribution function plot for proteomics data (all proteins) aggregated from 29 healthy human tissues (1) whereby transcripts were classified as uORF-containing or non-uORF-containing. The distribution of protein expression values is significantly lower for uORF-containing transcripts.

FIG. 3A shows analyses of uORF properties. Various functional mutants were generated to the HOXA11 uORF to change the strength of the Kozak context sequence, increase the length of the uORF, progressively truncate the uORF, and to add FLAG and HiBiT tags to the uORF. Nucleotide sequences are given in the sequence listing as SEQ ID NOs: 29-41 (numbered from top to bottom). Polypeptide sequences are given in the sequence listing as SEQ ID NOs: 42-50 (numbered from top to bottom; polypeptides with fewer than 4 amino acids have not been assigned a SEQ ID NO.) FIGS. 3B,C and D show cumulative distribution function plots for proteomics data taken from 29 healthy human tissues (1) whereby transcripts were classified as: (B) strong or weak Kozak contexts, (C) minimal uORF (ATG-STOP) or other uORFs, and (D) translation initiation site (TIS) spanning or not.

FIG. 4 is a schematic diagram showing regulatory element-containing exon skipping strategy. (A) Schematic diagram showing the structure of a hypothetical mRNA that contains a regulatory element in exon 2. This may be an upstream open reading frame (uORF) that represses the translational output of the primary ORF (pORF). Normal splicing of the pre-mRNA generates a mature mRNA that is subject to control by the regulatory element. (B) Antisense oligonucleotide (ASO) mediated exon skipping of a mRNA exon which contains a regulatory element. This results in exclusion of the regulatory element from the mature mRNA. If the regulatory element is a uORF, the resulting exon skipped-mRNA is de-repressed.

FIG. 5 is a schematic diagram showing regulatory element-containing exon inclusion strategy. (A) Schematic diagram showing the structure of a hypothetical mRNA with an alternatively spliced exon (or cryptic exon), labelled as ‘Exon 1b’, which is typically not spliced into the mature mRNA, and which contains a regulatory element. This regulatory element may be an upstream open reading frame (uORF) that has the potential ton represses the translational output of the primary ORF (pORF). Normal splicing of the pre-mRNA generates a mature mRNA that is not subject to control by the regulatory element. (B) Antisense oligonucleotide (ASO) mediated exon inclusion of a mRNA exon which contains a regulatory element. This results in inclusion of the regulatory element in the resulting mature mRNA. If the regulatory element is a uORF, the resulting exon included-mRNA is now repressed.

FIG. 6 is a schematic diagram showing regulatory element-containing exon skipping strategy applied to a long non-coding RNA (lncRNA). (A) Schematic diagram showing the structure of hypothetical a long non-coding RNA (lncRNA) that contains a regulatory element in exon 2. This may be a translated micropeptide that exhibits some function in the cell. Conversely, the regulatory element may be a domain that is important for the function of the lncRNA (e.g. it forms an RNA secondary structure that mediates an RNA:protein interaction). Normal splicing of the pre-lncRNA to generate the mature lncRNA results in production of the micropeptide/inclusion of the regulatory domain. (B) Antisense oligonucleotide (ASO) mediated exon skipping of a lncRNA exon which contains a regulatory element. This results in exclusion of that element from the mature lncRNA. If the regulatory element is a micropeptide, then this peptide is no longer generated by the exon-skipped mature lncRNA. If the regulatory element is a RNA:protein interaction domain, then this interaction will consequently be disrupted in the case of the exon-skipped mature lncRNA.

FIG. 7 shows the analysis of predicted uORFs at the BDNF locus. (A) Genome browser screenshot of the BDNF locus showing the 17 RefSeq transcript isoforms. The MANE Select Variant is indicated. NM_001143811 and NM_001143814 are two isoforms with skippable 5′ UTR exons. Positions of predicted uORFs are indicated and the data are combined with publicly available riboseq and RNA-seq data from the GWIPS-viz browser. (B) Zoomed in view of the first exons for 9 transcript isoforms with riboseq evidence of translation at certain uORFs.

FIG. 8 shows the effect of exon deletion on BDNF primary ORF translation. (A) Schematic of the 5′ UTR for the BDNF v11 transcript (NM_001143811) indicating the sizes and positions of exons, the locations of predicted uORFs, and the number of uORFs per exon. The pORF start codon is located in exon 4. (B) HEK293T cells were transfected with BDNF v11 5′ UTR-DLR wild-type and mutant constructs as indicated, and luciferase activity determined after 24 hours. For each mutant, the exon structure and number of functional uORFs (open circles) are indicated. HOXA11 WT and HOXA11 TTG (uORF disrupted) constructs were transfected in parallel as positive controls for uORF regulation and successful transfection. (C) RT-qPCR was used to determine RLuc transcript levels normalized to FLuc expression in parallel. (D) Schematic of the 5′ UTR for the BDNF v14 transcript (NM_001143814). The pORF start codon is located in exon 3. (E) HEK293T cells were transfected with BDNF v14 5′ UTR-DLR wild-type and ΔExon2 constructs as indicated, and luciferase activity determined after 24 hours. Values are mean+SEM, n=4 for DLR data and n=3 for RT-qPCR data. Very similar results were obtained from at least three independent repeats for each experiment. Differences between groups were tested by one-way ANOVA and Bonferroni post hoc test. ****P<0.0001.

FIG. 9 shows that uORFs are partially responsible for the repressive activity of BDNF v11 exon 2. (A) HEK293T cells were transfected with BDNF v11 (NM_001143811) 5′ UTR-DLR wild-type and mutant constructs as indicated, and luciferase activity determined 24 hours post transfection. Constructs were generated in which exon 2, exon 3, or both exons 2 and 3 were deleted. An additional construct was generated in which all 8 uORFs in exon 2 were disrupted by mutating the start codons to TTG. For each construct, the exon structure and number of functional or disrupted uORFs (open and closed circles, respectively) are indicated. HOXA11 WT and HOXA11 TTG (uORF disrupted) constructs were transfected in parallel as positive controls for uORF regulation and successful transfection. (B) Schematic of the BDNF v11 5′ UTR with the HuR #1 and HuR #2 motif sites indicated. The sizes and positions of exons, the locations of predicted uORFs, and the number of uORFs per exon are also indicated. (C) HEK293T cells were transfected with various BDNF v11 5′ UTR-DLR constructs. Mutants were generated in which either or both of the HuR motifs were deleted. An additional construct in which both motifs were deleted and all exon 2 uORFs were disrupted was tested in parallel. Luciferase activity was determined 24 hours post transfection. Values are mean+SEM, n=4. Very similar results were obtained from at least three independent repeats for each experiment. Differences between groups were tested by one-way ANOVA and Bonferroni post hoc test. ***P<0.001, ****P<0.0001, ns, not significant.

FIG. 10 shows deletion walk analysis of BDNF v1l exon 2. (A) HEK293T cells were transfected with BDNF v11 (NM_001143811) 5′ UTR-DLR wild-type and mutant constructs as indicated, and luciferase activity determined 24 hours post transfection. Constructs were generated in which 50 bp regions spanning exon 2 were sequentially deleted. HOXA11 WT and HOXA11 TTG (uORF disrupted) constructs were transfected in parallel as positive controls for uORF regulation and successful transfection. (B) HEK293T cells were treated as above with BDNF v11 constructs in which 10 bp regions spanning the first 50 bp of exon 2 were sequentially deleted. Values are mean+SEM, n=4. Very similar results were obtained from at least two independent repeats for each experiment. Differences between groups were tested by one-way ANOVA and Bonferroni post hoc test. *P<0.05, ****P<0.0001, ns, not significant.

### DETAILED DESCRIPTION OF THE INVENTION

### Regulatory Element

The invention relates to any regulatory element in a RNA transcript, where the regulatory element regulates the expression or activity of a gene of interest. For example, the regulatory element may regulate when, where and how much the gene is expressed in the form of RNA or protein, and/or its activity. For example, the regulatory element may be a translational regulatory element, a RNA processing regulatory element, a localisation element, an iron response element (IRE), a riboswitch, a miRNA recognition element, a RNA-binding protein recognition site, or a site of hybridisation with another endogenous RNA transcript.

The translational regulatory element may be an upstream open reading frame (uORF), or a secondary structure, such as a stem-loop, a hairpin, or a G-quadruplex.

The RNA processing regulatory element may be a splicing signal, an adenylate-uridylate-rich element (ARE) or a transcript stabilising motif.

The regulatory element may be a cis-regulatory element or a trans-regulatory element. Typically, the regulatory element is a cis-regulatory element. In one embodiment, the regulatory element is not a trans-regulatory element.

In the embodiment where the regulatory element is a trans-regulatory element, the regulatory element may be a micropeptide-encoding sequence, e.g. encoded within a long non-coding RNA (lncRNA). Micropeptides are polypeptides with a length of less than 100-150 amino acids that are encoded by short open reading frames, e.g. see reference 2. Hence, an exon comprising a micropeptide-encoding sequence may be skipped according the invention, such that no micropeptide can be translated.

The regulatory element may be anywhere in a RNA transcript. For example, if the RNA transcript is a protein-coding RNA transcript, the regulatory element may be in the 5′ untranslated region (UTR), such as an uORF. Alternatively, the regulatory element may be in the 3′ UTR, such as a miRNA binding site or an alternate polyadenylation signal. In one embodiment, the regulatory element is not a RNA processing regulatory element. In one embodiment, the regulatory element is not a splicing signal.

The regulatory element may be an uORF. uORFs are regulatory sequences which are present in 5′ UTRs and consist of a start codon and an in-frame stop codon. The presence of one or more uORFs in a RNA transcript is associated with translation repression of the downstream pORF. uORFs can also influence gene expression via other mechanisms, such as transcript stability via nonsense-mediated decay. Features of uORFs are known in the art and methods of identifying are within the skill of a person in the art (e.g. see references 3 and 4).

The inventors found that the addition and/or removal of one or more uORFs in a RNA transcript can significantly modulate the protein expression of the downstream pORF. In particular, the inventors performed functional mutagenesis on the RNA transcripts of three genes, BDNF, SCN1A and BRD3, each containing multiple uORFs in the 5′ UTR of the RNA transcript. Sequential deletion of each of these uORFs in turn demonstrated that uORF-mediated repression was additive, and that targeted disruption of a single uORF using RNA editing reduced the repressive effects on the downstream protein, and significantly increased protein expression.

Hence, the invention may involve introducing and/or removing one or more exons comprising one or more regulatory elements (e.g. uORFs), e.g. ≤50, ≤40, ≤30, ≤20, ≤10, ≤5, or 1 regulatory elements (e.g. uORFs). The regulatory elements may be located in one or more exons.

The inventors found that the more potent the regulatory element (e.g. uORF), the more effective the functional consequence (i.e. expression of the downstream pORF) when the regulatory element (e.g. uORF) is introduced and/or removed. Hence, the invention may comprise a step of identifying a regulatory element (e.g. uORF), such as a potent uORF, comprising one or more of the features described below. For example, the regulatory element (e.g. uORF) to be introduced and/or removed may be capable of reducing the expression of the protein it naturally regulates (e.g. downstream pORF) by ≥50%, ≥60%, ≥70%, ≥80%, ≥90%, or 100%.

An uORF useful with the invention may be within 500 nucleotides upstream of the pORF. For example, the regulatory element (e.g. uORF) may be ≤400, ≤300, ≤200, ≤100, ≤90, ≤80, ≤70, ≤60, ≤50, ≤30, ≤20, ≤10, ≤5 nucleotides upstream of the pORF start codon. For example, the uORF may be one that would have been within 100 nucleotides upstream of the pORF in a mature RNA transcript (after natural splicing of the RNA transcript has taken place).

The inventors found that the closer the uORF is to the pORF start codon in a mature RNA transcript (after natural splicing of the RNA transcript has taken place), the stronger its repressive effects, and so these uORFs would be useful targets of the invention. Hence, in a RNA transcript that comprises multiple uORFs, the invention may involve skipping at least the uORF that would have been the most proximal to the pORF start codon in a mature RNA transcript (after natural splicing of the RNA transcript has taken place).

An uORF useful with the invention may be of any length. The inventors found that uORFs consisting of the minimal sequence (START-STOP) are potent translational repressors, and so these uORFs would be useful targets of the invention. Hence, an uORF useful with the invention may comprise ≤50, ≤40, ≤30, ≤20, ≤10, ≤5, ≤4, ≤3, ≤2, 1, or 0 codons between the start codon and the stop codon. In a particular embodiment, the uORF may comprise ≤5, ≤4, ≤3, ≤2, 1, or 0 codons between the start codon and the stop codon.

An uORF useful with the invention may overlap with the pORF. For example, the uORF may be one that would have overlapped with the pORF in a mature transcript (after natural splicing of the RNA transcript has taken place). In this embodiment, splice modulation according to the invention results in the uORF being partially excised, whilst the pORF remains intact.

An uORF useful with the invention may not overlap with the pORF.

An uORF useful with the invention may overlap with another uORF.

An uORF useful with the invention may comprise a Kozak consensus sequence nnnnAUGn (SEQ ID NO: 16). The Kozak sequence may be a strong Kozak sequence comprising guanine at the +4 position and a purine at the −3 position (relative to the first nucleoside of the start codon, e.g. A of the AUG start codon). The Kozak sequence may be a weak Kozak sequence comprising a purine at the −3 position but not a guanine at the +4 position (relative to the first nucleoside of the start codon, e.g. A of the AUG start codon), and vice versa. The Kozak sequence may be a weak Kozak sequence which lacks both a purine at the −3 position and a guanine at the +4 position (relative to the first nucleoside of the start codon, e.g. A of the AUG start codon). For example, the uORF may comprise a Kozak sequence such as n[a/g]nnAUGg (SEQ ID NO: 17).

An uORF useful with the invention may comprise a higher percentage composition of acidic and basic amino acids as compared to aromatic hydrophobic amino acids.

A regulatory element useful with the invention may be present naturally in the RNA transcript. Alternatively, the regulatory element may be introduced by non-canonical and/or ectopic splicing events. Alternatively, the regulatory element may be introduced by mutations.

For example, single nucleotide polymorphisms (SNPs) or other mutations may introduce a regulatory element (e.g. uORF). In such embodiments, splice modulation according to the invention may be utilised to modulate expression of the mutant transcript with the aim of reversing the effects of the mutation-induced regulatory element (e.g. uORF). For example, Table 4 lists examples of genes with mutations or SNPs that create uORFs and their associated diseases.

In a further example, a cryptic exon containing a regulatory element (e.g. uORF) may be present in one or more of the introns of 5′ UTR of a RNA transcript. The cryptic exon may be present naturally in the wild-type RNA transcript, or may be introduced as a consequence of mutations. Non-canonical and/or ectopic splicing of the cryptic exon may introduce the cryptic exon in the RNA transcript. In such embodiments, splice modulation according to the invention may be utilised to remove the cryptic exon, thereby removing the inserted regulatory element (e.g. uORF).

Typically, the invention relates to modulating the presence a regulatory element in its entirety, such as the entire uORF from the start codon to stop codon. However, the invention may also relate to modulating the presence of a portion of the regulatory element. For example, the invention may relate to modulating the presence of a portion of the uORF, e.g. removing only the portion encoding the start codon of the uORF, whilst the remaining uORF remains in the RNA transcript. Hence, in the methods and uses of the invention, the regulatory element may be entirely removed or introduced, or partially removed or introduced.

### Splice Modulation

The invention relates to inducing splice modulation to skip and/or include one or more exons containing one or more regulatory elements (e.g. uORFs). Methods of inducing splice modulation for the treatment of diseases are known in the art (5). However, splice modulation approaches to date have typically been applied to the coding region of a RNA transcript, with the aim of restoring the reading frame of the RNA transcript such that a functional protein product is produced. In contrast, the invention relates to using splice modulation to alter the presence of regulatory elements in a RNA transcript.

In the embodiments of the invention where exon skipping in the 5′ UTR is involved, the target RNA transcript is one that would naturally have a spliced 5′ UTR in the mature RNA transcript (after natural splicing events have taken place). Hence, the RNA transcript contains, before natural splicing events have taken place, at least two exons upstream of the exon containing the pORF start codon (see FIG. 4). For example, exon-skipping may involve removing exon 2 and/or one or more downstream exons in the 5′ UTR. The exon containing the pORF start codon is not skipped.

The invention may involve skipping of a single exon or multiple exons. The regulatory elements may be located in one or more exons. Hence, the invention may involve skipping a single exon comprising one or more regulatory elements in the RNA transcript. Alternatively, the invention may involve skipping multiple exons comprising one or more regulatory elements in the RNA transcript.

The invention may involve exon inclusion of a single exon or multiple exons. The regulatory elements may be located in one or more exons. Hence, the invention may involve introducing a single exon comprising one or more regulatory elements in the RNA transcript. Alternatively, the invention may involve introducing multiple exons comprising one or more regulatory elements in the RNA transcript.

The invention may involve multi-exon splice modulation, i.e. skipping one or more exons and introducing one or more exons. Hence, the invention may involve skipping a single exon comprising one or more regulatory elements in the RNA transcript and introducing a single exon comprising one or more regulatory elements in the RNA transcript. Alternatively, the invention may involve skipping multiple exons comprising one or more regulatory elements in the RNA transcript and introducing multiple exons comprising one or more regulatory elements in the RNA transcript.

Multi-exon splice modulation may be achieved using a composition of two or more compounds (e.g. an antisense oligonucleotide) of the invention, as explained further below.

### Target Site and RNA Transcript

The invention involves targeting a splicing signal to induce splice modulation.

A splicing signal useful with the invention may comprise a splicing motif, such as a 5′ splice donor site, a 3′ splice acceptor site, an exon splicing enhancer sequence (ESE), a splicing branch point, a polypyrimidine tract, an intronic splicing silencer (ISS) sequence. Such splicing motifs are known in the art and can be identified using methods known in the art, e.g. bioinformatics techniques.

The 5′ splice donor site may comprise the sequence [C/A]AGgu[a/g]ag (SEQ ID NO: 18).

The 3′ splice acceptor site may comprise the sequence cagG[G/U] (SEQ ID NO: 19).

The exon splicing enhancers (ESEs) are motifs recognised by proteins of the SR family, which function to recruit components of splicing machinery to splice sites. An ESE useful with the invention may be a serine/arginine-rich splicing factor 1 (SRSF1) binding site (also known as SF2/ASF motif), a SC35 binding site, a SRp40 binding site, or a SRp55 binding site. For example, an ESE useful with the invention may be a SRSF1 binding site that comprises the sequence CACACGA (SEQ ID NO: 20). ESEs are well known in the art and can be identified by bioinformatics (e.g. see references 6,7).

The splicing branch point may comprise the sequence cu[a/g]A[c/u] (SEQ ID NO: 21).

In the embodiments of the invention, where the target site is a 5′ splice donor site, a 3′ splice acceptor site, an exon splicing enhancer sequence (ESEs), a splicing branch point, or a polypyrimidine tract, the compound (e.g. antisense oligonucleotide) of the invention may induce exon exclusion.

In the embodiments of the invention where an ISS sequence is targeted, the compound (e.g. antisense oligonucleotide) of the invention may induce exon inclusion.

Compounds of the invention may bind (e.g. hybridise) directly at the splicing signal. For example, the compound may hybridise fully or partially to the splicing signal. The compound of the invention typically does not bind away from the splicing signal.

The target site is typically devoid of RNA secondary structures.

A RNA transcript useful with the invention is typically a precursor messenger RNA (pre-mRNA). The pre-mRNA may not have undergone splicing. The pre-mRNA may have undergone partial splicing, i.e. a partially processed mRNA transcript. The RNA transcript may not be a mature mRNA.

The RNA transcript may be a protein-coding RNA transcript or a non-coding RNA transcript. A non-coding RNA transcript may be a long non-coding RNA (lncRNA), a long intervening non-coding RNA (lincRNA), or a macroRNA.

The RNA transcript is typically the natural transcript of a gene of interest.

Alternatively, the RNA transcript may be a chimeric RNA, e.g. resulting from aberrant genetic events or RNA processing events. For example, a chimeric RNA may arise from a fusion gene consisting of two genes which may have been joined through juxtaposition, often the result of a mutation (such as a chromosomal arrangement), e.g. the BCR-ABL fusion. As a further example, a chimeric RNA may arise as a result of two adjacent genes being transcribed on the same transcript and subsequently undergo splicing, such that their exons are joined together. In such cases, the compound, method or use of the invention may be utilised to modulate the expression of the fusion gene, or the expression or activity of the chimeric RNA.

The invention also provides a modified RNA transcript comprising the absence or inclusion of one or more exons comprising a regulatory element compared to the unmodified RNA transcript.

### Compound

A compound of the invention can cause activation of one or more splicing protein complexes in the cell to remove or introduce one or more exons from a RNA transcript. The compound may inhibit a protein that regulates splicing activity. The compound may activate a protein that regulates splicing activity. The compound may prevent one or more spliceosome components from recognising and/or accessing the splice motifs.

The compounds of the invention are not designed to elicit cleavage of the target RNA transcript. Whilst not wishing to be bound by theory, the compound of the invention induces steric block of a target sequence, and in such a way that it does not induce target cleavage via RNase H recruitment. Hence, in certain embodiments of the invention, the compound of the invention does not induce or has a reduced ability to induce RNase H cleavage of the target nucleic acid.

The compounds of the invention are designed such that it does not result in effects which act against the intended effects on gene expression or activity. For example, if the up-regulation of gene expression is desired, a compound of the invention is designed such that the induced splice modulation does not result in the introduction or formation of a negative regulatory element (e.g. uORF).

The compound is typically an oligonucleotide. In some embodiments, the compound may be a small molecule (e.g. having a molecular weight of less than 900 Da). Alternatively, the compound may be a polypeptide, e.g. an antibody.

The oligonucleotide comprises a plurality of linked nucleosides, e.g. DNA or RNA. The oligonucleotide may be a modified oligonucleotide, i.e. it comprises at least one modified nucleoside (e.g. at least one modified sugar moiety and/or at least one modified nucleobase moiety) and/or at least one modified internucleoside linkage. The modified oligonucleotide may be an antisense oligonucleotide, a nucleic acid aptamer, a Triplex-Forming Oligonucleotide (TFO) and a polypurine reverse-Hoogsteen hairpin.

In a preferred embodiment, the oligonucleotide may be a modified oligonucleotide. The modified oligonucleotide may be an antisense oligonucleotide.

The oligonucleotide (e.g. antisense oligonucleotide) may be up to 50, 40, 30, 20, 10 or 5 nucleotides in length. The oligonucleotide (e.g. antisense oligonucleotide) may be at least 5, 10, 15, 20, 25, 35 or 40 nucleotides in length. For example, the oligonucleotide (e.g. antisense oligonucleotide) may be between 5 to 40 nucleotides, between 10 to 40 nucleotides, 18 to 30 nucleotides, or between 13 to 25 nucleotides in length.

The oligonucleotide (e.g. antisense oligonucleotide) comprises a region that is sufficiently complementary to the target nucleic acid to allow hybridisation under physiological conditions. The oligonucleotide (e.g. antisense oligonucleotide) comprises a sequence complementary to the target site (explained above). The oligonucleotide (e.g. antisense oligonucleotide) may be fully or partially complementary to the target site. For example, the oligonucleotide (e.g. antisense oligonucleotide) may have ≥50%, ≥60%, ≥70%, ≥80%, ≥90%, ≥91%, ≥92%, ≥93%, ≥94%, ≥95%, ≥96%, ≥97%, ≥98%, ≥99% or 100% sequence complementarity to a target site.

The oligonucleotide (e.g. antisense oligonucleotide) may comprise mismatched regions internally within the oligonucleotide and/or at the termini of the oligonucleotide.

The oligonucleotide (e.g. antisense oligonucleotide) may comprise ≥3, ≥4, ≥5, ≥6, ≥7, ≥8, ≥9, ≥10, ≥11, ≥12, ≥13, ≥14, ≥15, ≥16, ≥17, ≥18, ≥19, and/or ≥20 contiguous complementary bases of the target site.

The oligonucleotide may comprise or consist of any of SEQ ID NOs: 22 to 26.

In embodiments where the oligonucleotide (e.g. antisense oligonucleotide) comprises a sequence complementary to a 5′ splice donor site, the oligonucleotide may comprise or consist of:

(SEQ ID NO: 22)
5′-[N]ₐGARUGGAM[N]_{b}-3′

where:
N is any nucleoside, or modified nucleoside thereof;
R is adenosine (A) or guanosine (G); or modified nucleoside thereof;
M is adenosine (A) or cytidine (C); or modified nucleoside thereof;
a is 0 to 27; and
b is 0 to 27.

In embodiments where the oligonucleotide (e.g. antisense oligonucleotide) comprises a sequence complementary to a 3′ splice acceptor site, the oligonucleotide may comprise or consist of:

(SEQ ID NO: 23)
5′-[N]ₐKGGAC[N]_{b}-3′

where:
N is any nucleoside; or modified nucleoside thereof;
K is guanosine (G) or uridine (U); or modified nucleoside thereof;
a is 0 to 27; and
b is 0 to 27.

In embodiments where the oligonucleotide (e.g. antisense oligonucleotide) comprises a sequence complementary to SF2/ASF motif, the oligonucleotide may comprise or consist of:

(SEQ ID NO: 24)
5′-[N]ₐUCGUGUG[N]_{b}-3′

where:
N is any nucleoside; or modified nucleoside thereof;
a is 0 to 27; and
b is 0 to 27.

In embodiments where the oligonucleotide (e.g. antisense oligonucleotide) comprises a sequence complementary to a splicing branch point, the oligonucleotide may comprise or consist of:

(SEQ ID NO: 25)
5′-[N]ₐRUYAG[N]_{b}-3′

where:
N is any nucleoside, or modified nucleoside thereof;
Y is cytidine (C) or uridine (U); or modified nucleoside thereof;
R is adenosine (A) or guanosine (G); or modified nucleoside thereof;
a is 0 to 27; and
b is 0 to 27.

In embodiments where the oligonucleotide (e.g. antisense oligonucleotide) comprises a sequence complementary to a polypyrimidine tract, the oligonucleotide may comprise or consist of:

(SEQ ID NO: 26)
5′-[N]ₐ[R]_{b}[N]_{c}

where:
N is any nucleotide, or modified or derivative thereof;
R is guanosine (G) or adenosine (A); or modified nucleoside thereof;
a is 0 to 27; and
b is 0 to 27.
c is 0 to 27.

The oligonucleotide (e.g. antisense oligonucleotide) may have a GC content of ≥40%, ≥50%, ≥60%. For example, the oligonucleotide (e.g. antisense oligonucleotide) may have a GC content between 40-60%.

The oligonucleotide (e.g. antisense oligonucleotide) may contain overhangs, whereby part of the sequence binds to the target transcript (with partial or full complementarity with respect to the target recognition domain) and also sequence overhangs (of up to 100 nucleotides) on one or both termini of the oligonucleotide. These overhangs may facilitate recruitment of cellular proteins (i.e. splicing factors) by forming aptameric structures (for example), or assist with oligonucleotide delivery.

Typically, the oligonucleotide (e.g. antisense oligonucleotide) may be single-stranded, but the oligonucleotide (e.g. antisense oligonucleotide) may also be partially or fully double-stranded.

The oligonucleotide (e.g. antisense oligonucleotide) may comprise or consist of a nucleic acid sequence having ≥70%, ≥80%, ≥90%, ≥91%, ≥92%, ≥93%, ≥94%, ≥95%, ≥96%, ≥97%, ≥98%, ≥99%, or 100% identity to a sequence selected from: SEQ ID NOs: 1 to 15, and optionally wherein the uracil nucleotides are substituted with thymine nucleotides. Examples of oligonucleotides useful with the invention are provided in Tables 1 and 2.

### Oligonucleotide Chemistry

The oligonucleotides discussed herein may be modified oligonucleotides. Modifications to the oligonucleotide are well known to the skilled person to impart useful properties, e.g. increase the biological stability of the molecules (e.g. nucleases resistance), enhance target binding, increase tissue uptake and/or increase the physical stability of the duplex formed between the oligonucleotide and target nucleic acids (e.g. see reference 8).

Typically, the oligonucleotide (e.g. antisense oligonucleotide) induces steric block of a target sequence, and in such a way that it does not induce target cleavage via RNase H recruitment. For example, the oligonucleotide (e.g. antisense oligonucleotide) may comprise a chemistry which does not support RNase H cleavage (i.e. do not generate consecutive runs of DNA or DNA-like bases), e.g. see Reference 9. For example, the oligonucleotide (e.g. antisense oligonucleotide) may comprise a ‘mixmer’ pattern in which the oligonucleotide may comprise 2 or more different nucleic acid chemistries, but runs of more than 2 or 3 DNA or DNA-like bases (which would support RNase H-mediated cleavage) are avoided.

The oligonucleotide (e.g. antisense oligonucleotide) may comprise DNA, RNA, and/or nucleotide analogues. The nucleotide analogues may be peptide nucleic acid (PNA), FANA, DANA, LNA, and other branched nucleic acids (ENA, cEt), phosphorodiamidate morpholino oligomer (PMO), and/or tricyclo DNA.

The oligonucleotide (e.g. antisense oligonucleotide) may comprise an abasic site, i.e. the absence of a purine (adenine and guanine) or a pyrimidine (thymine, uracil and cytosine) nucleobase.

The oligonucleotide (e.g. antisense oligonucleotide) may comprise a 3′ to 5′ phosphodiester (PO) linkage as naturally found in DNA or RNA. The oligonucleotide may comprise a modified internucleoside linkage, e.g. a phosphotriester linkage, a phosphorothioate (PS) linkage, a boranophosphate linkage, a phosphorodiamidate linkage, a phosphoamidate linkage, and/or a thiophosphoramidate linkage. The modified internucleoside linkage may be other modifications known in the art.

The oligonucleotide may comprise one or more asymmetric centres and thus give rise to enantiomers, diasteromers, and other stereoisomeric configurations, e.g. R, S. For example, stereochemistry may be constrained at one or more modified internucleoside linkages. For example, the oligonucleotide may comprise repeated left-left-right (or SSR) chiral PS centers.

The oligonucleotide (e.g. antisense oligonucleotide) may comprise a sugar moiety as found in naturally occurring RNA (i.e. a ribofuranosyl) or a sugar moiety as found in naturally occurring DNA (i.e. a deoxyribofuranosyl). The oligonucleotide may comprise a modified sugar moiety, i.e. a substituted sugar moiety or a sugar surrogate. Substituted sugar moiety moieties include furanosyls comprising substituents at the 2′-position, the 3′-position, the 5′-position and/or the 4′-position. A substituted sugar moiety may be a bicyclic sugar moiety (BNA). Sugar surrogates include morpholino, cyclohexeynl and cyclohexitol.

The modified sugar moiety may comprise a 2′-O-methyl, 2′-O-methoxyethyl (2′-O-MOE), 2′-O-aminopropyl, 2′-deoxy, 2′-O-propyl (2′-O-AP), 2′-O-dimethylaminoethyl (2′O-DMAOE), 2′-O-dimethylaminopropyl (2′-O-DMAP), 2′-O-dimethylaminoethyloxyethyl (2′O-DMAEOE), or 2′O—N-methylacetoamido (2′O-NMA) modification or a locked or bridged ribose conformation (e.g. LNA, cEt or ENA). The modified sugar moiety may comprise other modifications known in the art.

The oligonucleotide (e.g. antisense oligonucleotide) may comprise a terminal modification at its 5′ and/or 3′ end, such as a vinyl phosphonate, and/or inverted terminal bases.

The oligonucleotide (e.g. antisense oligonucleotide) may comprise a nucleobase as found in naturally occurring RNA and DNA (i.e. adenine (A), thymine (T), uracil (U), guanine (G), cytosine (C), inosine (I), and 5-methyl C). The oligonucleotide may comprise a modified nucleobase, e.g. 5-hyrdoxymethylcytosine, 5-formylcytosine, and 5-carboxycytosine. The inclusion of 5′methylcytosine may enhance base pairing by modifying the hydrophobic nature of the oligonucleotide.

The oligonucleotide (e.g. antisense oligonucleotide) may comprise a single type of nucleic acid chemistry (e.g. full PS-MOE, or full PMO) or combinations of different nucleic acid chemistries.

For example, each of the sugar moieties in the oligonucleotide (e.g. antisense oligonucleotide) may comprise a 2′-O-methoxyethyl (2′MOE) modification and each of the internucleoside linkages may be a phosphorothioate (i.e. a fully PS-MOE oligonucleotide). PS modifications are known to result in resistance to a broad spectrum of nucleases and increase protein binding, which also improves tissue uptake (10,11). 2′MOE modifications are known to enable enhanced binding affinity to the target mRNA with minimal toxicity and reduce plasma protein binding.

The oligonucleotide (e.g. antisense oligonucleotide) may be a fully phosphorodiamidate morpholino oligomer (PMO). Morpholinos are known to provide greater target affinity and facilitate nuclease avoidance (12).

The oligonucleotide (e.g. antisense oligonucleotide) may comprise a combination of PO and PS internucleoside linkages. This may facilitate the fining tuning of the pharmacokinetics of the oligonucleotide.

The oligonucleotide (e.g. antisense oligonucleotide) may be constructed using chemical synthesis and/or enzymatic ligation reactions using procedures known in the art. Exemplary methods can include those described in reference 13, 14, 15, 16, 17, 18, 19 or 20.

Alternatively, the oligonucleotide (e.g. antisense oligonucleotide) may be produced biologically using an expression vector into which the oligonucleotide is sub-cloned in an antisense orientation (i.e. RNA transcribed from the inserted oligonucleotide will be of an antisense orientation to the target nucleic acid of interest).

A compound of the invention may be an expressed exon skipping trigger, such as a small nuclear RNA (snRNA)-based trigger (e.g. U7 snRNA or U1 snRNA). Expressed splice modulation systems for facilitating alternative splicing of upstream exons may be delivered via plasmid or viral vectors (e.g. adenovirus-associated viral vector (AAV) or lentivirus).

### Conjugate

A compound of the invention may be conjugated to one or more further compounds, such as a nucleic acid molecule, a peptide, or other chemicals for the purpose of improving targeting (e.g. to a specific tissue, cell type, or cell developmental stage), improving cell penetration (e.g. delivery), improving endosomal escape, improving sub-cellular localisation, improving activity and/or promoting recruitment of a cellular protein. The compounds may be conjugated by any means known in the art, e.g. they may be chemically attached to the further compound via cleavable or non-cleavable linkers.

For example, a conjugated compound (e.g. conjugated oligonucleotide) of the invention may comprise an antisense oligonucleotide of the invention conjugated to a further antisense oligonucleotide of the invention. Each of the conjugated compounds may target a different site on the same RNA transcript.

The further compound may be a peptide, such as a cell penetrating peptide, a protein transduction domain, a targeting peptide, an endosmolytic peptide. The peptide conjugated to the compound of the invention may comprise a splicing factor to enhance, inhibit or modulate splicing.

The further compound may not target a splicing signal in the 5′ UTR of the RNA transcript.

The further compound may be a small molecule ligand (e.g. having a molecular weight of less than 900 Da).

The further compound may be an antibody, e.g. nanobody, Fab fragment.

The further compound may be a sugar-based ligand, e.g. GalNAc, or its derivatives).

The further compound may be a lipid-based ligand, e.g. cholesterol, lipidoid, lipid-like conjugate, lipophilic molecule.

The further compound may be a polymer (e.g. PEI, dendrimer).

The further compound may be a polyethylene glycol, a click-reactive group or an endosmolytic group (e.g. chloroquine or its derivatives).

The further compound may be a RNA molecule, e.g. an aptamer or any structure that enhances, inhibits or modulates splicing.

The compound of the invention may be combined as part of a platform molecule, e.g. a dynamic polyconjugate.

The compound (e.g. antisense oligonucleotide) of the invention may be conjugated to a delivery vehicle. Hence, the invention also provides a delivery vehicle comprising the compound (e.g. antisense oligonucleotide) of the invention. The delivery vehicle may be capable of site-specific, tissue-specific, cell-specific or developmental stage-specific delivery.

The delivery vehicle may comprise a lipid-based nanoparticle, a cationic cell penetrating peptide (CPP), a linear or branched cationic polymer, or a bioconjugate, such as cholesterol, bile acid, lipid, peptide, polymer, protein, or an aptamer.

For example, the delivery vehicle may comprise an antibody, or part thereof. The antibody may be specific for a cell surface marker on the cells of interest for delivery of the compound of the invention to the specific cells. For example, the specific cells may be beta cells in the pancreas, thymic cells, malignant cells, and/or pre-malignant cells (e.g. pre-leukaemias and myelodysplastic syndromes or histopathologically defined precancerous lesions or conditions).

The delivery vehicle may comprise a cell penetrating peptide (CPP). Suitable CPPs are known in the art, e.g. as described in reference 21. For example, the CPP may be an arginine and/or lysine rich peptide. Hence, the CPP may comprise a poly-L-lysine (PLL) and/or a poly-arginine. The CPP may comprise a Pip peptide. Advantageously, a Pip peptide conjugate has a high potency and can reach cardiac muscle following systemic delivery. The delivery vehicle may comprise a peptide-based nanoparticle (PBN), wherein a plurality of CPPs form a complex with the polynucleic acid polymer through charge interactions.

The delivery vehicle may comprise a nanoparticle. Advantages of nanoparticles include bespoke optimisation of nanoparticle biophysical properties such as size, shape, material and ligand functionalisation for targeting. Examples of suitable nanoparticles include, lipoplexes, liposomes, exosomes, spherical nucleic acids, and DNA nanostructures (e.g. DNA cages).

The compound of the invention may be complexed with (e.g. by ionic bonding) or covalently bound to a delivery vehicle. Suitable conjugation methods are known in art, e.g. as described in reference 22. For example, a conjugation method may comprise introducing a suitable tether containing a reactive group (e.g. —NH₂ or —SH₂) to the compound of the invention and to the delivery vehicle (e.g. a peptide) post-synthetically as an active intermediate, followed by carrying out the coupling reaction in aqueous medium. An alternative method may comprise carrying out the conjugation in a linear mode on a single solid-phase support.

### Polynucleotide and Vector

The invention also provides a polynucleotide encoding an oligonucleotide or conjugated oligonucleotide according to the invention.

Polynucleotides which encode an oligonucleotide or conjugated oligonucleotide of the invention can be obtained by methods well known to those skilled in the art. General methods by which the vectors may be constructed, transfection methods and culture methods are well known to those skilled in the art, e.g. see 23.

A polynucleotide of the invention may be provided in the form of an expression cassette, which includes control sequences operably linked to the inserted sequence, thus allowing for expression of the oligonucleotide or conjugated oligonucleotide of the invention in vivo. Hence, the invention also provides one or more expression cassettes encoding the one or more polynucleotides that encoding an oligonucleotide or conjugated oligonucleotide of the invention. These expression cassettes, in turn, are typically provided within vectors. Hence, in one embodiment, the invention provides a vector encoding an oligonucleotide or conjugated oligonucleotide of the invention. The vector may be a vector for cloning purposes (e.g. a plasmid). The vector may be a vector for expression of the polynucleotide in a cell.

The vector may be a viral vector, such as an adeno-associated viral vector (AAV) or lentiviral vector. The vector may comprise any virus that targets the oligonucleotide or conjugated oligonucleotide according to the invention to a specific cell type.

The polynucleotide, expression cassette or vector of the invention is introduced into a host cell. Hence, the invention also provides a host cell comprising a polynucleotide, expression cassette or vector of the invention. The polynucleotide, expression cassette or vector of the invention may be introduced transiently or permanently into the host cell, allowing expression of an oligonucleotide or conjugated oligonucleotide from the expression cassette or vector.

### Composition

The invention provides a composition comprising a compound (e.g. an antisense oligonucleotide), a conjugated compound (e.g. a conjugated antisense oligonucleotide), a polynucleotide or a vector of the invention. The composition may comprise a combination (such as 2, 3, 4, 5, 6, 7, 8, 9 or 10) of the compounds (e.g. antisense oligonucleotides) of the invention. Each compound may be targeted to a different (but possibly overlapping) sequence of the same RNA transcript. Alternatively, each compound may be targeted to a different RNA transcript.

The composition may be a pharmaceutical composition. A pharmaceutical composition of the invention may comprise a pharmaceutically acceptable excipient, carrier, buffer, stabiliser or other materials well known to those skilled in the art. Such materials are typically non-toxic and does not interfere with the efficacy of the active ingredient. The precise nature of the carrier or other material may be determined by the skilled person according to the route of administration.

In one embodiment, the pharmaceutical composition comprises a sterile saline solution (e.g. PBS) and one or more antisense compounds of the invention.

The composition of the invention may include one or more pharmaceutically acceptable salts, esters or salts of such esters. A pharmaceutically acceptable salt refers to a salt that retains the desired biological activity of the parent compound and does not impart any undesired toxicological effects. Examples of such salts include sodium or potassium salts.

The compound of the invention may be in the form of a prodrug. The prodrug may include the incorporation of additional nucleosides at one or both ends of an oligonucleotide which are cleaved by endogenous nucleases when administered, to form the active compound.

The pharmaceutical composition may comprise lipid moieties. For example, the oligonucleotide (e.g. antisense oligonucleotide) of the invention is introduced into preformed liposomes or lipoplexes made of mixtures of cationic lipids and neutral lipids. The lipid moiety may be selected to increase distribution of the oligonucleotide to a particular cell or tissue, e.g. fat tissue or muscle tissue.

The pharmaceutical composition may comprise a compound (e.g. antisense oligonucleotide) and one or more excipients. The excipient may be water, salt solutions, alcohol, polyethylene glycols, gelatin, lactose, amylase, magnesium stearate, talc, silicic acid, viscous paraffin, hydroxymethylcellulose and/or polyvinylpyrrolidone.

The pharmaceutical composition may comprise a delivery system, such as liposomes and emulsions. In certain embodiments, organic solvents such as dimethylsulfoxide are used.

The pharmaceutical composition may comprise one or more tissue-specific delivery molecules designed to deliver the one or more compounds (e.g. antisense oligonucleotides) of the invention to specific tissues or cell types. For example, the delivery molecule may comprise liposomes coated with a tissue-specific antibody.

For delayed release, a vector may be included in a pharmaceutical composition which is formulated for slow release, such as in microcapsules formed from biocompatible polymers or in liposomal carrier systems according to methods known in the art.

Pharmaceutical compositions of the invention may comprise additional active agents, for example a drug or a pro-drug.

The pharmaceutical composition may be formulated to be administered by any administration route, e.g. as described herein. The pharmaceutical composition is typically administered by injection. In such embodiments, the pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives).

### Method and Use

The methods and uses of the invention may be in vitro, ex vivo or in vivo. For example, the invention also provides an in vitro or ex vivo method for modulating the presence of a regulatory element in a RNA transcript, comprising delivering to a cell a compound targeted to a splicing signal in the RNA transcript to induce splice modulation of one or more exons comprising the regulatory element.

Hence, in some embodiments, the method or use of the invention is not a treatment of the human or animal body by surgery or therapy and is not a diagnostic method practised on the human or animal body.

The invention further relates to the use of the compound (e.g. antisense oligonucleotide), conjugated compound, polynucleotide or vector encoding the compound, or the composition described herein, e.g. in a method of therapy practiced on the human or animal body.

For example, the invention relates to a method of treating or preventing a disease or condition in a subject by modulating the expression or activity of a gene, comprising administering to the subject a therapeutically effective amount of the compound (e.g. antisense oligonucleotide) or the composition of the invention.

The invention also relates to the use of the compound (e.g. antisense oligonucleotide) or the composition of the invention in the manufacture of a medicament for the treatment or prevention of a disease or condition in a subject by modulating the expression of a gene.

The invention also relates to the compound (e.g. antisense oligonucleotide) or the composition of the invention for use in a method of treating or preventing a disease or condition in a subject by modulating the expression or activity of a gene.

The invention also relates to the compound (e.g. antisense oligonucleotide) or the composition of the invention for a method of treating or preventing a disease or condition in a subject by modulating the expression or activity of a gene.

The methods and uses of the invention may comprise inhibiting the disease state, e.g. arresting its development; and/or relieving the disease state, e.g. causing regression of the disease state until a desired endpoint is reached. The methods and uses of the invention may comprise the amelioration or the reduction of the severity, duration or frequency of a symptom of the disease state (e.g. lessen the pain or discomfort), and such amelioration may or may not be directly affecting the disease.

For instance, the invention relates to a method of treating or preventing a disease listed in Table 3 or 4. Hence, the methods and uses of the invention comprise administering to the subject a therapeutically effective amount of the compound or the composition of the invention a compound of the invention, wherein the compound is targeted to the RNA transcript of the respective gene listed in Table 3 or 4.

The RNA transcript may be encoded by a uORF-containing gene, such as: ABCA1, ABCB11, ABCC2, ABCG5, ADAM10, ALB, ANK1, APOE, ATP2A2, ATP7B, ATRX, ATXN1, ATXNIL, BAX, BCL2L11, BDNF (e.g. BDNF v11), BLM, BRCA1, C/EBPα, CA2, CASP8, CCBE1, CD36, CD3D, CDKN1B, CDKN2A, CEP290, CFH, CFTR, CHRNA4, CHRNA5, CNTF, CNTFR, COL1A1, CR1, CSPP1, CTNND2, CTNS, CYP1B1, DBT, DCAF17, DNASE1, DDIT3, DICER1, DRD3, EED, EFNB1, EPO, ESR1, ETHE1, EZH2, F8 (and F2, 3, 5, 7, 11, 13), FAP, FMR1, FNDC5, FXN, GALNS, GATA3, GBA, GCH1, GCK, GH2, GRN, HBB, HBD, HBE1, HBG1, HBG2, HCRT, HGF, HNF4a, HR, HSD17B4, IDO1, IFNE and other interferon genes, IFRD1, IGF1, IGF1R, IGF2, IGF2BP2, IGFBP3, IGHMBP2, IL6, INS, IQGAP1, IQGAP2, IRF6, IRS2, ITGA7, JAG1, KCNJ11, KCNMA1, KCNMB1, KCNMB2, KCNMB3, KCNQ3, KLF4, KMT2D, LDLR, LRP1, LRP5, LRP8, LRPPRC, MBTPS1, MECP2, MSRA, MSX2, MTR, MUTYH, MYCN, MYF6, NAMPT, NANOG, NEU4, NF1, NKX2, NKX3, NKX5, NKX8, NOD2, NR5A, NRF1, NSD1, PAH, PARK2, PKD1, PLAT, PON1, PON2, PPARD, PRKARIA, PRPF31, PTEN, PYCR1, RB1, RBL1, RBL2, RBBP4, RNASEH1, ROR2, RPS14, RPS19, SCNIA, SCN2A, SERPINF1, SERPING1, SHBG, SIRT1, SLC1A2, SMAD7, SMCHD1, SMN1, SMN2, SNX27, SPINK1, SRB1, SRY, ST7, ST7L, STAT3, TFE3, TFEB, TGFB3, THPO, TP63, TP73, UCP2, USP9Y/SP3, UTRN or VEGFA.

The RNA transcript may be encoded by an isoform of a uORF-containing gene, such as BDNF v11.

The RNA transcript may be encoded by a gene with mutations or SNPs that create one or more uORFs, such as: ATP7B, ATRX, BLM, BRCA1, CA2, CCBE1, CD3D, CD4, CDKN2A, CFL2, CFTR, CSPP1, CTNS, DBT, DCAF17, DCLREIC, DFNB31, DLG4, DMD, DNASE1, ETHE1, GALNS, GCH1, HAMP, HBB, HMBS, HR, IGHMBP2, IRF6, ITGAZ, ITGB2, KCNJ11, KCNQ3, LDLR, LRP5, LRP5L, MECP2, MLH1, MSH6, MUTYH, NR5A1, PALB2, PANK2, PEX7, PHYH, PIK3R5, POMC, POMT1, ROR2, SCN2A, SGCA, SGCD, SLC16A1, SLC19A3, SLC2A2, SLC7A9, SPINK1, SRY, STIL, TK2, TMPRSS3, TP53, TPI1, TPM3, TRMU, TSEN54, or ZEB1.

The RNA transcript may be a long non-coding RNA (lncRNA).

The methods and uses of the invention may comprise increasing, decreasing, or restoring the expression of a protein of interest by splice modulation of its RNA transcript.

Splice modulation to induce the inclusion and/or exclusion of specific exons in the 5′ UTR of a RNA transcript, and hence the regulatory elements contained therein, has functional consequences for the downstream pORF.

Hence, the invention also provides a method of increasing, decreasing or restoring the amount of expression or activity of a target gene, comprising a method of inducing alternative splicing of one or more exons as described herein.

In the embodiments of the invention where gene expression or activity is increased, the gene expression or activity may be increased by ≥50% (i.e. 50% or more), ≥60%, ≥70%, ≥80%, ≥90%, ≥100% or ≥200% compared to the gene expression or activity in cells which have not been in contact with a compound of the invention.

In the embodiments of the invention where gene expression or activity is reduced, the gene expression or activity may be reduced by ≥50% (i.e. 50% or more), ≥60%, ≥70%, ≥80%, ≥90% or 100% compared to the gene expression or activity in cells which have not been in contact with a compound of the invention.

The methods and uses of the invention may include a step of determining the expression and/or activity level of the RNA transcript which is modulated by splicing (e.g. mature mRNA) and/or the protein encoded by the RNA transcript in a sample from the patient. Methods of determining the expression and/or activity levels of RNAs and proteins are known in the art. For example, RNA from a sample may be isolated and tested by hybridisation or PCR techniques as known in the art. Alternatively, protein expression assays can be performed in vivo, in situ, i.e. directly upon tissue sections (fixed and/or frozen) of patient tissue obtained from biopsies or resections, such that no nucleic acid purification is necessary. Immunoassays may also be used, e.g. Western Blot or ELISA.

The RNA transcript may be encoded by a gene listed in Table 3 or 4. The diseases associated with each gene in Table 3 may be treated or prevented by the methods of the invention using a compound of the invention targeting the RNA transcript of the respective gene.

The methods and uses of the invention relate to delivering a compound of the invention to a cell. The cell may be a eukaryotic cell, e.g. a human cell. The cell may be from non-human animals such as mice, rats, rabbits, sheep, pigs, cows, cats, or dogs is also contemplated.

Typically, the invention relates to methods and uses for a human subject in need thereof. However, non-human animals such as mice, rats, rabbits, sheep, pigs, cows, cats, or dogs are also contemplated.

The invention relates to analysing samples from subjects. The sample may be tissues, cells and biological fluids isolated from a subject, as well as tissues, cells and fluids present within a subject. The sample may be blood and a fraction or component of blood including blood serum, blood plasma, or lymph.

The protein detection assays may be performed in situ, in which case the sample is a tissue section (fixed and/or frozen) of the tissue obtained from biopsies or resections from a subject.

The compound or composition of the invention may be administered subcutaneously, intravenously, intradermally, orally, intranasally, intramuscularly, intracranially, intrathecally, intracerebroventricularly, intravitreally, or topically (e.g. in the form of a cream for skin).

Dosages and dosage regimes appropriate for use with the invention can be determined within the normal skill of the medical practitioner responsible for administration of the composition. For example, for treatment purposes, a therapeutically effective amount of the compound or composition of the invention would be administered to such a subject. A therapeutically effective amount is an amount which is effective to ameliorate one or more symptoms of the disorder.

The dosage may be determined according to various parameters, especially according to the age, weight and condition of the patient to be treated; the nature of the active ingredient, the route of administration; and the required regimen. A physician will be able to determine the required route of administration and dosage for any particular patient.

For example, an antisense oligonucleotide of the invention may be administered at a dose of between about 1 mg/kg and about 300 mg/kg, such as about 50 mg/kg, by intramuscular injection.

The dose may be provided as a single dose, but may be repeated (e.g. for cases where vector may not have targeted the correct region and/or tissue (such as surgical complication)).

The compound or composition of the invention may be administered in a multiple dosage regimen. For example, the initial dose may be followed by administration of a second or plurality of subsequent doses. The second and subsequent doses may be separated by an appropriate time.

The compound or composition of the invention are typically used in a single pharmaceutical composition/combination (co-formulated). However, the invention also generally includes the combined use of the compound or composition of the invention in separate preparations/compositions. The invention also includes combined use of the compound or composition of the invention with additional therapeutic agents, as described herein.

Combined administration of the two or more agents may be achieved in a number of different ways. In one embodiment, all the components may be administered together in a single composition. In another embodiment, each component may be administered separately as part of a combined therapy.

For example, the compound or composition of the invention may be administered before, after or concurrently with another compound or composition of the invention.

The invention also provides kits and articles of manufacture for use with the invention. The kit may comprise a compound (e.g. an antisense oligonucleotide), a conjugated compound (e.g. a conjugated antisense oligonucleotide), a polynucleotide, a vector, a delivery vehicle, a composition or a pharmaceutical composition of the invention and instructions for use. The kit may further comprise one or more additional reagents, such as buffers necessary for the makeup and delivery of the compound (e.g. antisense oligonucleotide) of the invention. The kit may further comprise package inserts with instructions for use.

### Other

It is to be understood that the terminology used herein is for the purpose of describing particular embodiments of the invention only, and is not intended to be limiting.

In addition as used in this specification and the appended claims, the singular forms “a”, “an”, and “the” include plural references unless the content clearly dictates otherwise. Thus, for example, reference to “a regulatory element” includes two or more regulatory elements.

Furthermore, when referring to “≥x” herein, this means equal to or greater than x.

The term “comprising” encompasses “including” as well as “consisting” e.g. a composition “comprising” X may consist exclusively of X or may include something additional e.g. X+Y.

For the purpose of this invention, in order to determine the percent identity of two sequences (such as two polynucleotide or two polypeptide sequences), the sequences are aligned for optimal comparison purposes (e.g. gaps can be introduced in a first sequence for optimal alignment with a second sequence). The nucleotide or amino acid residues at each position are then compared. When a position in the first sequence is occupied by the same nucleotide or amino acid as the corresponding position in the second sequence, then the nucleotides or amino acids are identical at that position. The percent identity between the two sequences is a function of the number of identical positions shared by the sequences (i.e. % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 3, SEQ ID NO: 3 would be the reference sequence. To assess whether a sequence is at least 95% identical to SEQ ID NO: 3 (an example of a reference sequence), the skilled person would carry out an alignment over the length of SEQ ID NO: 3, and identify how many positions in the test sequence were identical to those of SEQ ID NO: 3. If at least 95% of the positions are identical, the test sequence is at least 95% identical to SEQ ID NO: 3. If the sequence is shorter than SEQ ID NO: 3, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

As used herein, “complementary” in reference to oligomeric compounds means the capacity of such oligomeric compounds or regions thereof to hybridize to another oligomeric compound or region thereof through nucleobase complementarity under stringent conditions. For example, in DNA, adenine (A) is complementary to thymine (T). For example, in RNA, adenine (A) is complementary to uracil (U). Nucleobases comprising certain modifications may maintain the ability to pair with a counterpart nucleobase and thus, are still capable of nucleobase complementarity.

Percent complementarity means the percentage of nucleobases of an oligomeric compound that are complementary to an equal-length portion of a target nucleic acid. Percent complementarity of two oligomeric compounds can be determined by aligning them for optimal comparison purposes (e.g. mismatches or gaps can be introduced in a first sequence for optimal alignment with a second sequence) and comparing the nucleobases at each position. Percent complementarity can be calculated by dividing the number of nucleobases of the oligomeric compound that are complementary to nucleobases at corresponding positions in the target nucleic acid by the total length of the oligomeric compound.

The nucleic acid sequences in the sequence listing accompanying this application identifies each sequence as either “RNA” or “DNA” as required. However, one of skill in the art would appreciate such sequences in the sequence listing to also describe modified oligonucleotides, i.e. these sequences may also represent oligonucleotides having any combination of modifications described herein. For example, an oligonucleotide having the sequence “GAATGGAC” encompasses any oligonucleotides having such nucleobase sequences, whether modified or unmodified, such as oligonucleotides having RNA bases, e.g. “GAAUGGAC”, and/or oligonucleotides having other modified or naturally occurring bases, such as “GAAUGGA^{m}C” where ^{m}C is 5-methylcytosine.

All publications, patents and patent applications cited herein, whether supra or infra, are hereby incorporated by reference in their entirety.

The following examples illustrate the invention.

### EXAMPLES

### Example 1

Prediction of uORFs in the Human and Mouse Transcriptomes

In eukaryotic mRNAs, the primary open reading frame (pORF) is often preceded by one or more upstream open reading frames (uORFs). These uORFs are highly diverse in terms of sequence length, number of uORFs per transcript, distance from the 5′ m7G-cap, distance from the pORF, strength of uORF Kozak sequence, evolutionary conservation, and whether the uORF overlaps with the pORF. uORFs can be predicted either computationally or empirically observed. The aim of this experiment is to identify uORFs in human and mouse protein coding genes, and the results are explained below.

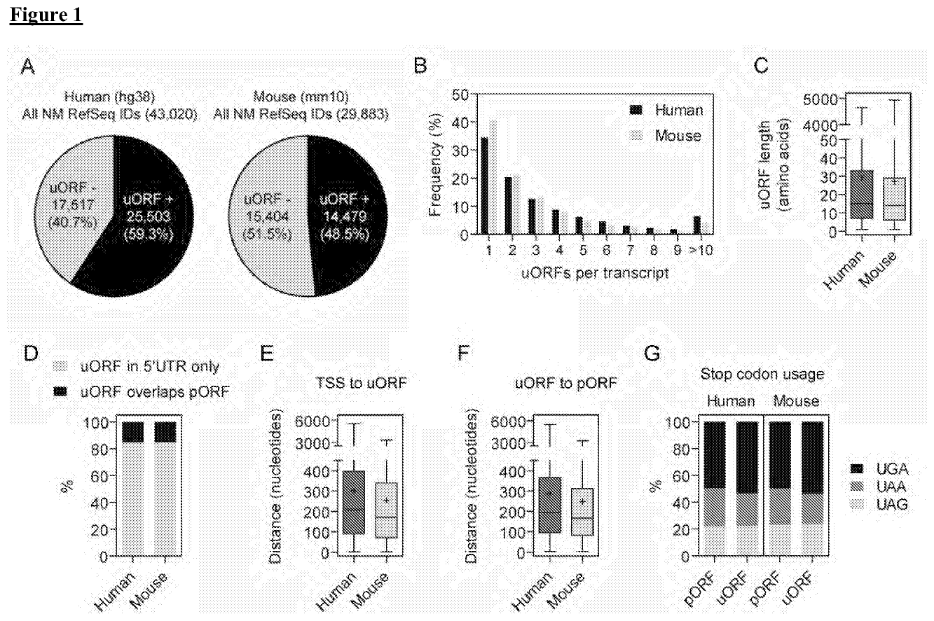

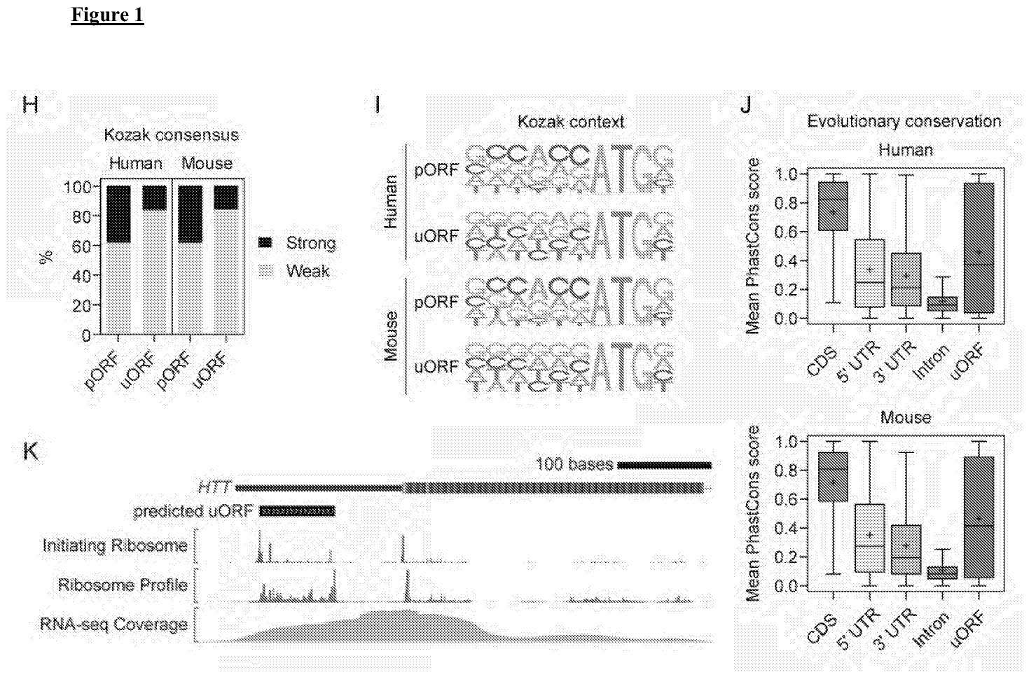

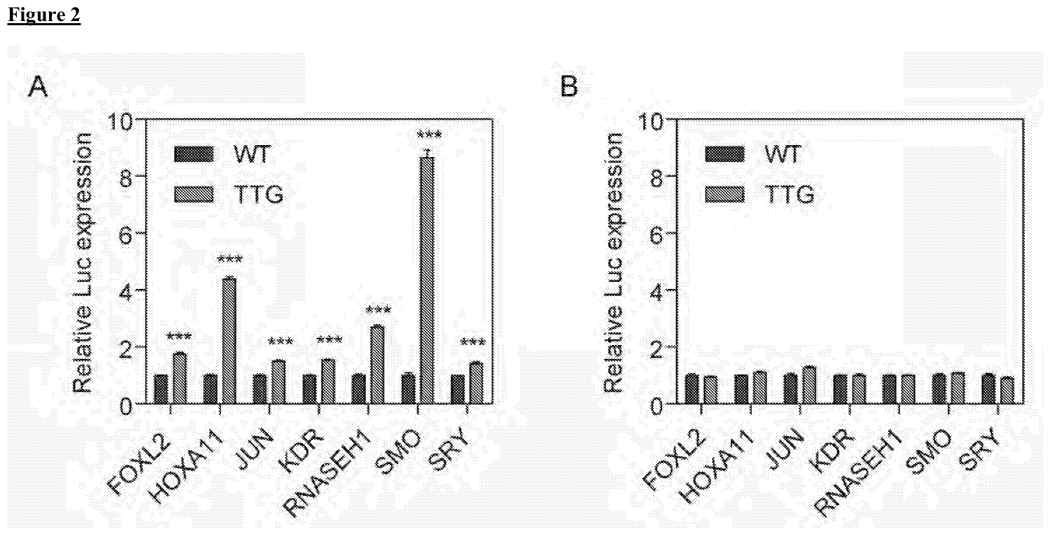

Predicted uORFs were identified in the 5′ UTRs of all human and mouse protein-coding genes using a custom script. 59.3% of human transcripts and 48.5% of mouse transcripts were found to have at least one predicted uORF (FIG. 1A), which was comparable to previous estimates using similar approaches with earlier genome builds (24,25,26,27). For those transcripts with predicted uORFs, the majority contained only a single uORF, although ˜6% of human and ˜4% of mouse transcripts contained 10 or more uORFs (FIG. 1B). The majority of uORFs were between 6 and ˜30 amino acids in length (FIG. 1C), and ˜85% of uORFs did not overlap with the pORF (FIG. 1D). The distance between the transcription start site (TSS) and the start of the uORF (FIG. 1E), as well as between the uORF and the pORF (FIG. 1F), was ˜100-400 nt for the majority of transcripts in each group, and ranged from 1 to ˜6,000 nt for the human genome, and 1 to ˜3000 nt for the mouse genome (FIGS. 1E and F). The most commonly utilized stop codon was similar between pORFs and uORFs, with UGA being the most frequent (FIG. 1G).

The Kozak consensus sequence was generally weaker for uORFs than for pORFs (FIG. 1H,I). Genomic coordinates (i.e. BED format) were calculated for predicted uORFs which enabled average phastCons scores to be determined for each uORF (and compared with 5′ UTRs, 3′ UTRs, CDS, and intronic regions). Conservation values for uORF sequences were highly diverse, essentially spanning the full range of possible phastCons values, suggesting that some are highly conserved, while others are poorly conserved (FIG. 1J). Overall, predicted uORF properties were highly similar between human and mouse (FIG. 1A-J).

uORF predictions were visualized using the GWIPS-viz genome browser (28) together with aggregated ribosome profiling and RNA-seq data, which allows for the identification of uORFs for which there is experimental evidence. The HTT gene is shown as an example (FIG. 1K) whereby a prominent initiating ribosome peak and ribosome footprint are observed at the predicted uORF.

Validation of Predicted uORF Functionality

Predicted human uORF-containing genes were tested by cloning the corresponding 5′ UTR upstream of Renilla luciferase in a dual luciferase reporter system. For each candidate gene, control constructs were generated in which the uORF was disrupted by mutagenesis of the uATG to TTG. The relative levels of Renilla and Firefly luciferase where analysed for each 5′ UTR and mutant controls by RT-qPCR.

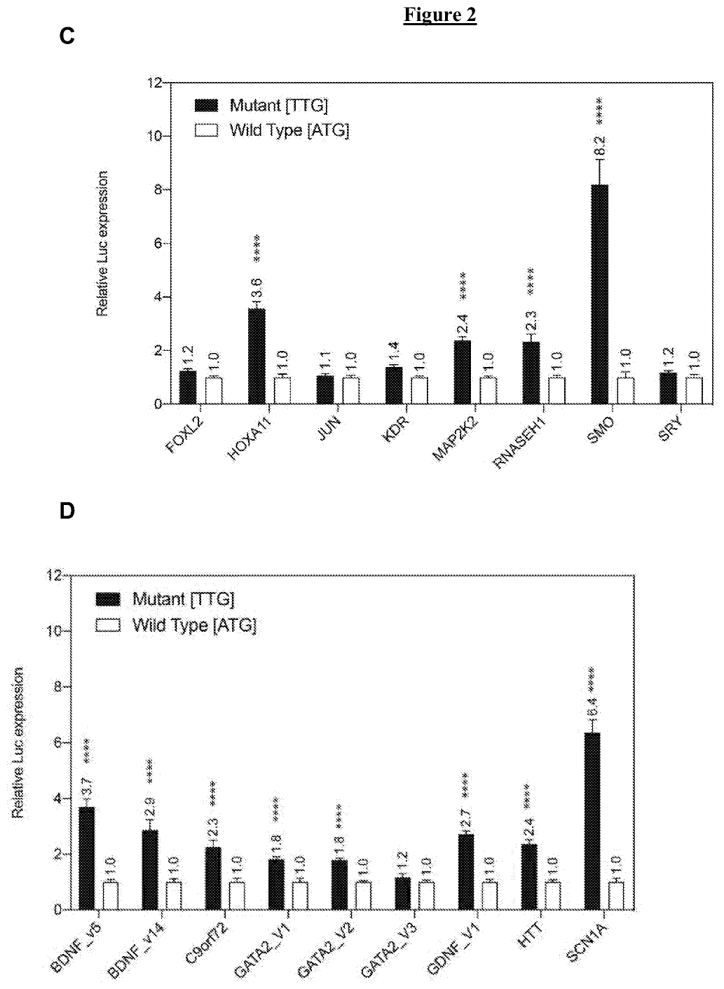

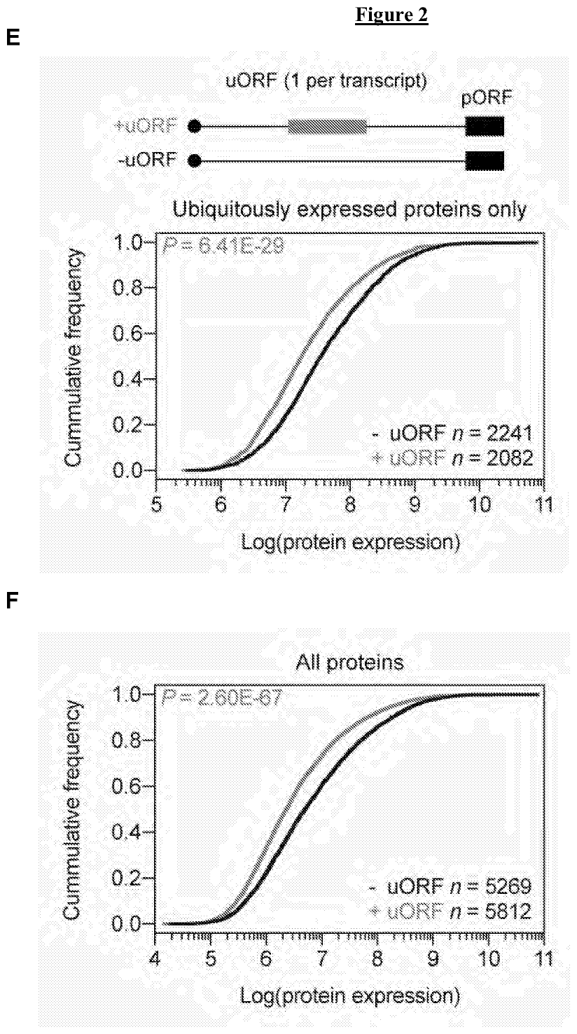

The results are shown in FIG. 2. A significant increase in luciferase expression was observed for FOXL2, HOXA11, JUN, KDR, RNASEH1, SMO and SRY (FIG. 2A) which could not be explained by changes in transcript levels (FIG. 2B). Luciferase data were independently re-produced and an additional gene, MAP2K2, added to the panel (FIG. 2C). Significant uORF-mediated repression was further observed in reporter constructs of BDNF (2 isoforms), C9orf72, GATA2 (3 isoforms), GDNF, HTT and SCN1A when all the predicted uORFs were mutated from ATG to TTG (FIG. 2D).

To assess the impact of uORF sequences on translational repression on a global scale, publicly available, matched proteomics/transcriptomics data from 29 healthy human tissues (1) were utilised. These data enable the study of 18,072 transcripts and 13,640 proteins. Data were filtered to exclude genes with multiple transcript isoforms where at least one of those isoforms lacked a uORF, and the remaining data binned as uORF containing or uORF-lacking respectively. The cumulative distribution of protein expression data (aggregated across all tissues) for all genes in each bin were then plotted and the difference between the distributions tested by Mann-Whitney test for ubiquitously expressed proteins (FIG. 2E) or all proteins (FIG. 2F). It was observed that genes containing uORFs have a significantly lower median protein expression levels than genes lacking uORFs.

Analysis of uORF Structure and Function

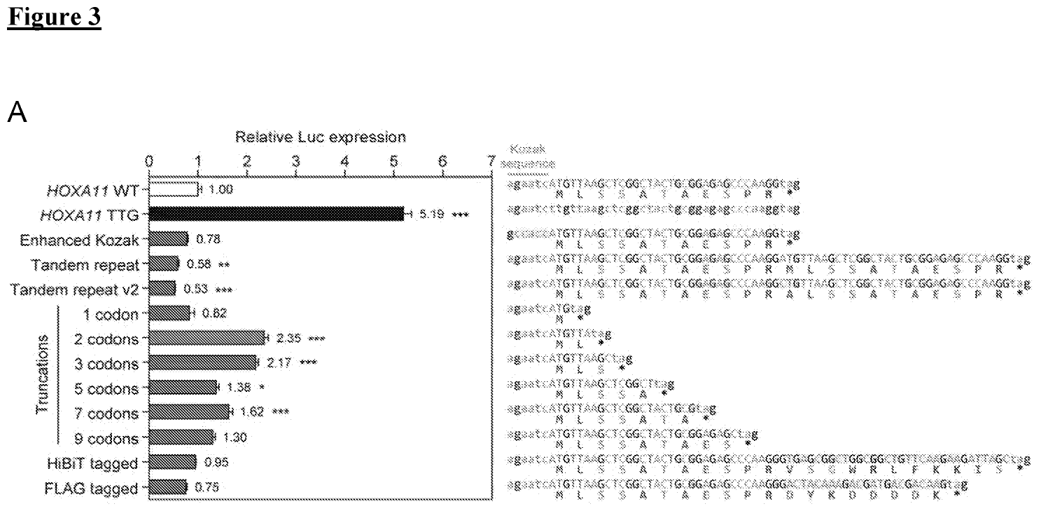

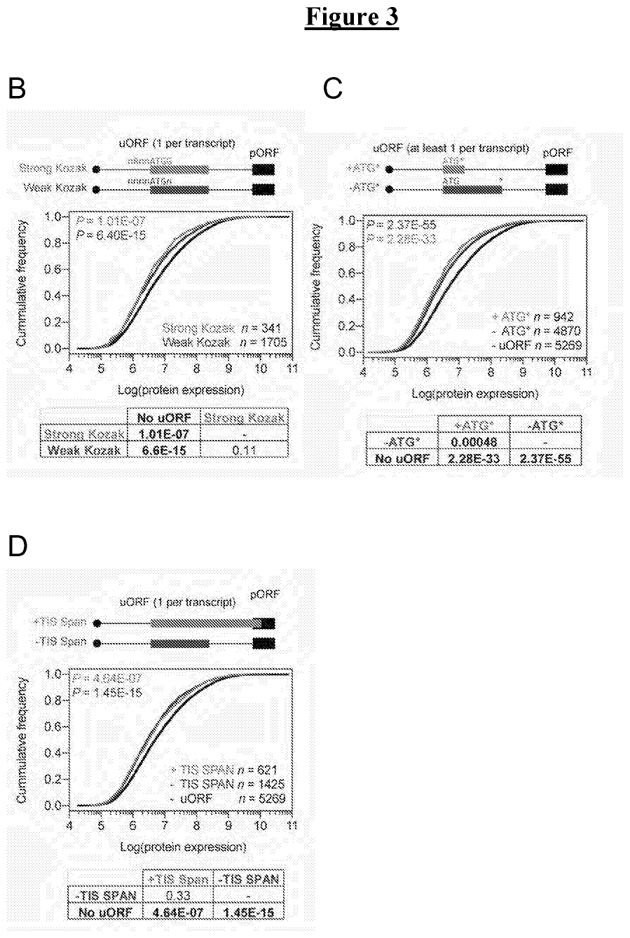

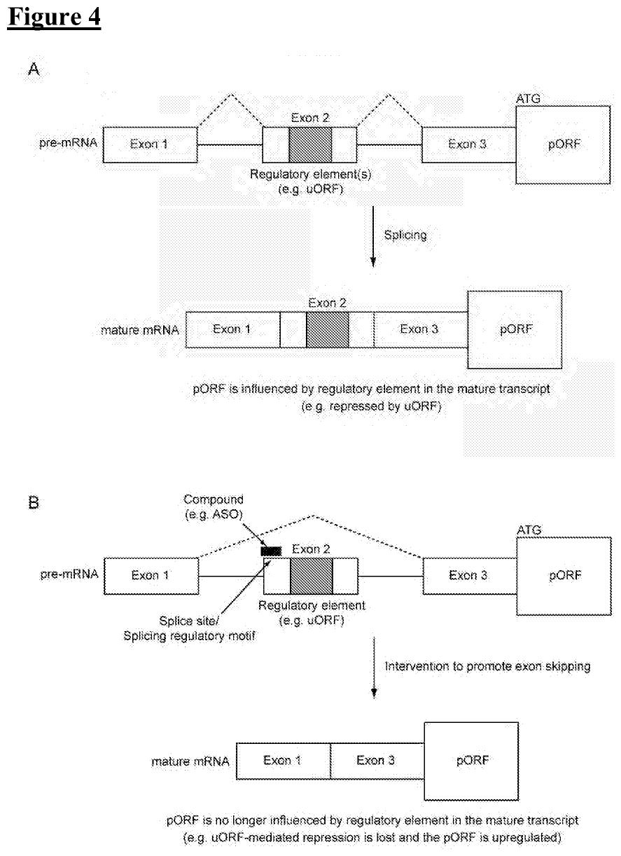

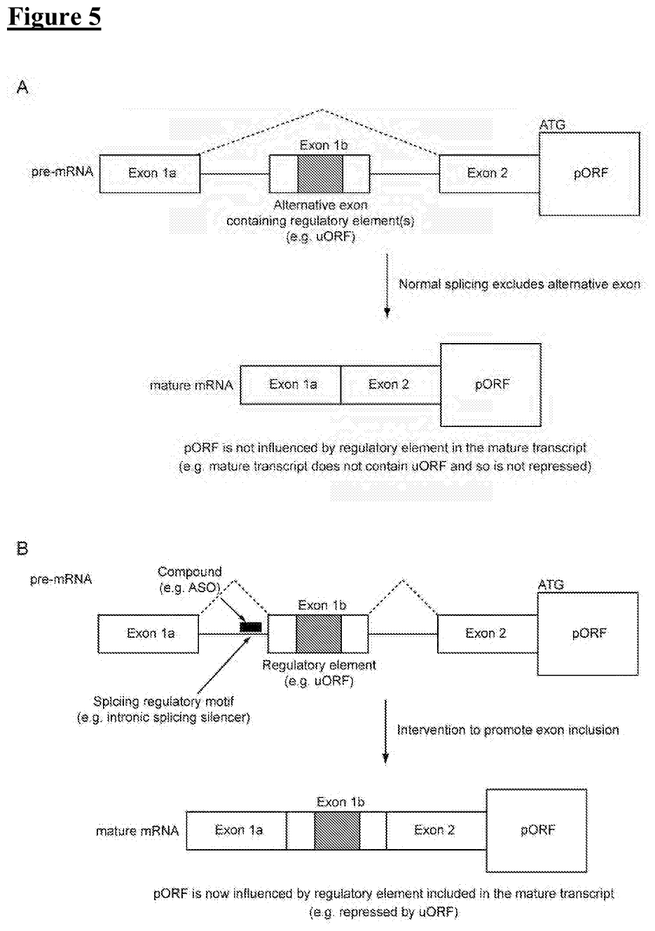

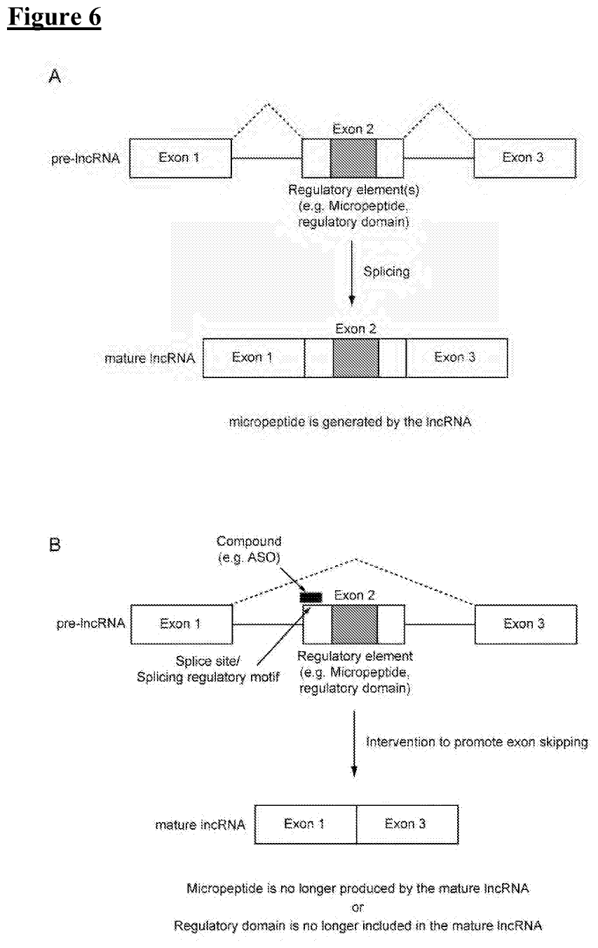

The HOXA11 uORF was selected for further study, as this uORF conferred a strong repressive translational repressive effect (˜4 fold) in reporter studies, and the length of both the 5′ UTR and uORF were of convenient lengths for facile experimental manipulation. A plasmid was constructed in which the HOXA11 uORF was replaced with a cloning site, and a variety of mutants of the wild-type HOXA11 uORF were subsequently generated. Altering the Kozak consensus at the HOXA11 uORF uATG resulted in an enhancement of downstream gene repression by 22% which did not reach statistical significance at the P<0.05 level (FIG. 3A). Consistently, global analysis of publicly-available protein expression data revealed no difference in median expression between transcripts containing ‘weak’ (n=1,729) or ‘strong’ (n=350) Kozak contexts at the predicted uORFs, suggesting that Kozak context strength is not a major factor in determining uORF activity (FIG. 3B). This analysis is complicated by the relatively low number of transcripts containing a single uORF with a strong Kozak consensus sequence. Notably, the findings are in contrast with a previous report which utilized a mouse proteomics dataset, although in this study only 92 transcripts were included with ‘strong’ uORF Kozak contexts (24).

Next, the importance of uORF peptide length was investigated through a series of C-terminal truncations from the C-terminus (i.e. to generate uORFs that were 9, 7, 5, 3, 2, 20 and 1 amino acids in length). Progressive truncation of the HOXA11 uORF resulted in a partial loss of repressive activity that was proportional to the degree of truncation. The minimal uORF (i.e. ATG-STOP, M*, methionine-STOP) did not fit this pattern and instead exhibited repression that was not statistically different from the wild-type HOXA11 uORF (FIG. 3A). It has been reported that an unoccupied ribosome E site results in translational repression (29). This is suggestive of at least two mechanisms of translational repression. Notably, such minimal uORFs occur frequently in in the prediction datasets; 5,447 (6% of all uORFs) in human, and 2,759 (6.5%) in mouse.

Global analysis of aggregated human proteomics data revealed that if all uORF-containing transcripts, those which contained the minimal uORF (n=802) tended to be expressed at lower levels than those that contained other types of uORFs (n=4,991) (P=0.0027) (FIG. 3C).

Increasing the length of the uORF by repeating the entire amino acid sequence resulted in a statistically significant ˜40% increase in repressive activity. However, extending the length of the uORF by the addition of tag sequences (i.e. FLAG and HiBiT) had only minimal effects on the degree of translational repression (FIG. 3A).

Aggregate proteomics data showed that there was no difference in repressive potential between uORFs which span the TIS (n=816) and those that are completely contained within the 5′ UTR (n=1,652) (FIG. 3D), consistent with previous observations 6. Experimental validation of TIS-spanning uORFs is complicated if the pORF is replaced with a reporter gene (as described herein).

### Example 2

In this example, antisense oligonucleotides (ASOs) are designed to target a splicing signal in the 5′ UTR of the RNA transcript of ACY3, CLGN or SYS1, with the aim of removing an exon containing at least one uORF. The effects of these ASOs on the expression of the ACY3, CLGN or SYS1 protein are investigated.

Each of ACY3, CLGN, and SYS1 contains three upstream exons where the pORF start codon was located in exon 3, and at least 1 uORF was located in exon 2.

The sequences of some example ASOs are provided in Table 1. Each of the ASOs is a fully phosphorothioate RNA—with fully 2′MOE modifications.

TABLE 1
Examples of ASOs for inducing exon skipping in 5′UTR of ACY3, CLGN or
SYS1.

| | | | SEQ ID |
|---|---|---|---|
| Gene | Target site | Sequence 5′ to 3′ | NO: |
| ACY3 | Branch point | TGGGTGGGACGGGCTGAGGTTCATG | 1 |
| ACY3 | Py tract | GGCAAGCTGGCAGACAGCAGGG | 2 |
| ACY3 | Splice acceptor | ATTCATGGGCCTGGAGATCCA | 3 |
| ACY3 | SRSF1 site | TTCCCGGGCCACCAGGACTG | 4 |
| ACY3 | Splice donor | GGGGTACTTACCGCTGATGC | 5 |
| CLGN | Branch point | TAAAATCAGCGAAAGTGTCTGAT | 6 |
| CLGN | Py tract | TTGCTTGGGCAGATGCTATAAA | 7 |
| CLGN | Splice acceptor | TTTGGTTGCCATTTGCTTTTAACTC | 8 |
| CLGN | SRSF1 site | CAAACACCTCCTCTTTGTTGCTT | 9 |
| CLGN | Splice donor | TGGCCACGTTATTTACCTTTTCTCT | 10 |
| SYS1 | Branch point | CTCCTAGTTAGAGTCTGATAAC | 11 |
| SYS1 | Py tract | GGAAAGCAGCGGAGGGGCGG | 12 |
| SYS1 | Splice acceptor | CGGCAGGAGCGGCTGCGTAG | 13 |
| SYS1 | SRSF1 site | GGCAAAGCTCCAGCGACCAC | 14 |
| SYS1 | Splice donor | GTCTACTCACCAGTGACAGACT | 15 |

The ASOs are administered to mammalian cells in culture, or injected into human patients or animal models. The ASOs are injected by intramuscular injection into human patients or animal models. The ASOs are injected in a sterile buffer (e.g. saline) at a dose of about 50 mg/kg.

The amount and/or activity of the ACY3, CLGN or SYS1 protein is determined prior to and after administration of the ASOs to cells or subjects.

Each of the ASOs induces skipping of exon 2 in the RNA transcript of ACY3, CLGN or SYS1, and leads to an increase in the protein expression and/or activity of ACY3, CLGN or SYS1 compared to control (where cells or subjects are administered with mock ASOs).

### Example 3

In this example, antisense oligonucleotides (ASOs) are designed to target a long non-coding RNA (lncRNA), with the aim of removing an exon containing a sequence that encodes a micropeptide. The effects of these ASOs on the expression of the micropeptide are investigated.

The lncRNA HOXB-AS3 encodes a short peptide involved in colon cancer. The sequence of this peptide starts in exon 2 of the HOXB-AS3 transcript (e.g. NR_033201.2 and NR_033204.2). An ASO designed to skip exon 2 of this transcript would prevent translation of this short peptide.

Table 2 shows some example ASOs targeted to the splicing signal of exon 2 of HOXB-AS3. Each of the ASOs is a fully phosphorothioate RNA—with fully 2′MOE modifications.

TABLE 2
Examples of ASOs for inducing exon skipping in lncRNA.

| IncRNA | Target site | Sequence 5′ to 3′ | SEQ ID NO: |
|---|---|---|---|
| HOXB- | ESE | TCTCCGCCGAGGCCGGCGAG | 27 |
| AS3- | | | |
| peptide | | | |
| HOXB- | Splice | GAGGAAACGGCTAGAGAAAC | 28 |
| AS3- | acceptor | | |
| peptide | | | |

The ASOs are administered to mammalian cells in culture, or injected into human patients or animal models. The ASOs are injected by intramuscular injection into human patients or animal models. The ASOs are injected in a sterile buffer (e.g. saline) at a dose of about 50 mg/kg.

The amount and/or activity of the micropeptide is determined prior to and after administration of the ASOs to cells or subjects.

Each of the ASOs induces skipping of exon 2 in the lncRNA, and leads to the lack of the micropeptide expression compared to control (where cells or subjects are administered with mock ASOs).

### Example 4

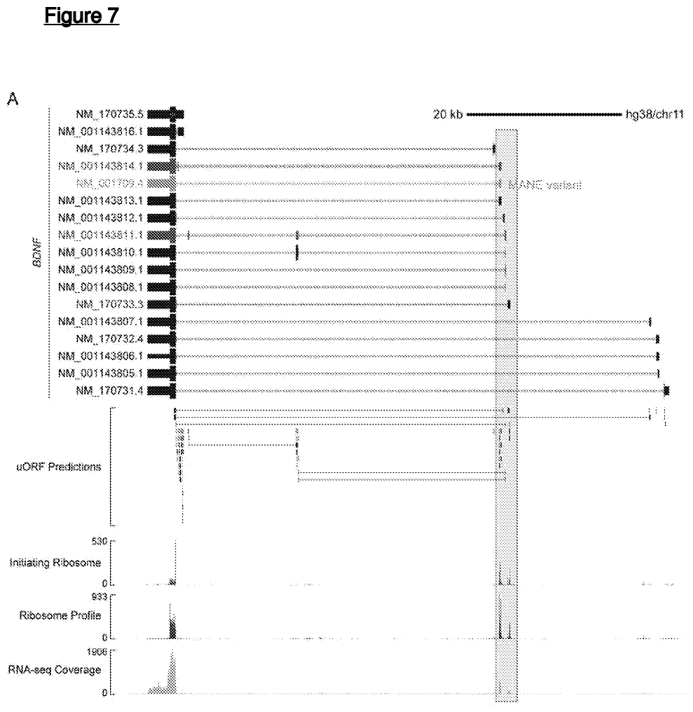

In this example, the human BDNF locus was analysed to identify predicted uORFs with riboseq/RNA-seq data overlaid (FIG. 7).

Two BDNF isoforms were identified that qualified as putative exon skipping targets: transcript variant 11 (NM_001143811) and transcript variant 14 (NM_001143814). These transcripts contained at least three exons in the 5′ UTR, with the skippable exon containing at least one uORF (FIG. 7).

The 5′ UTRs of each these transcripts were cloned downstream of a Renilla luciferase gene as part of an in-house dual luciferase reporter plasmid, and variants generated whereby each skippable exon, or combination of exons, was deleted. All constructs were transfected in HEK293T cells and luciferase activity measured 24 hours post transfection.

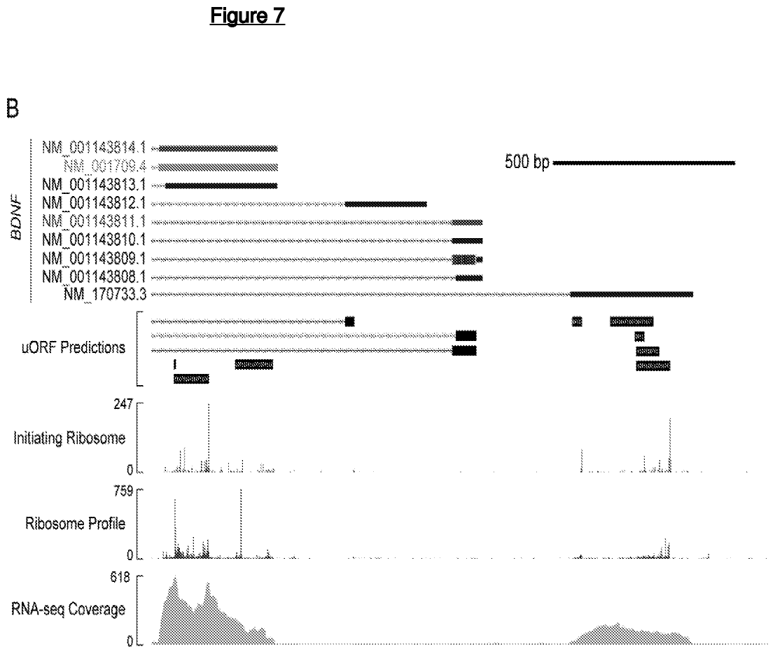

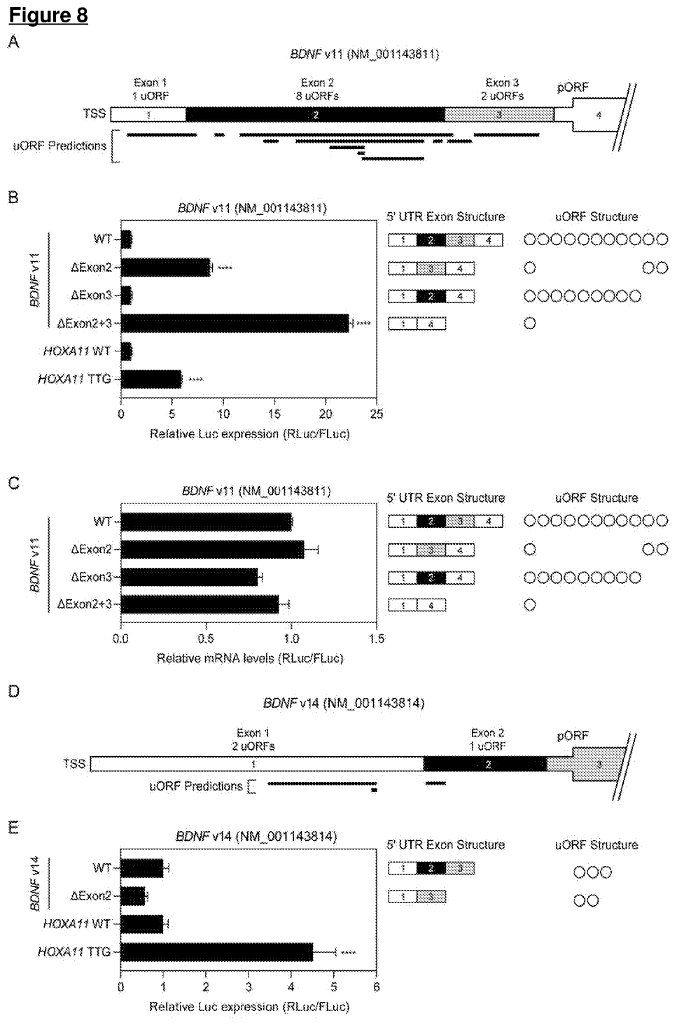

For BDNF v11, deletion of exon 2 (containing 8 predicted uORFs) resulted in a ˜8-fold upregulation of pORF reporter gene expression (FIG. 8A,B). Deletion of both exons 2 and 3 resulted in even further de-repression, with a ˜23-fold increase in Renilla luciferase activity. However, deletion of exon 3 alone (containing 2 predicted uORFs) did not affect pORF reporter gene expression. The profound protein level upregulation could not be explained by changes in transcript levels as there was no difference between groups (FIG. 8C).

These data suggest that exon skipping strategies which aim to exclude exon 2 or exons 2 and 3 from the mature BDNF transcript could potentially be exploited for therapeutic BDNF upregulation at the level of translation.

In contrast, analysis of BDNF v14 showed that when exon 2 was deleted (containing 1 uORF) there was no significant effect on reporter gene expression (FIG. 8D,E). These data suggest that this transcript variant is a less suitable target for upstream exon skipping.

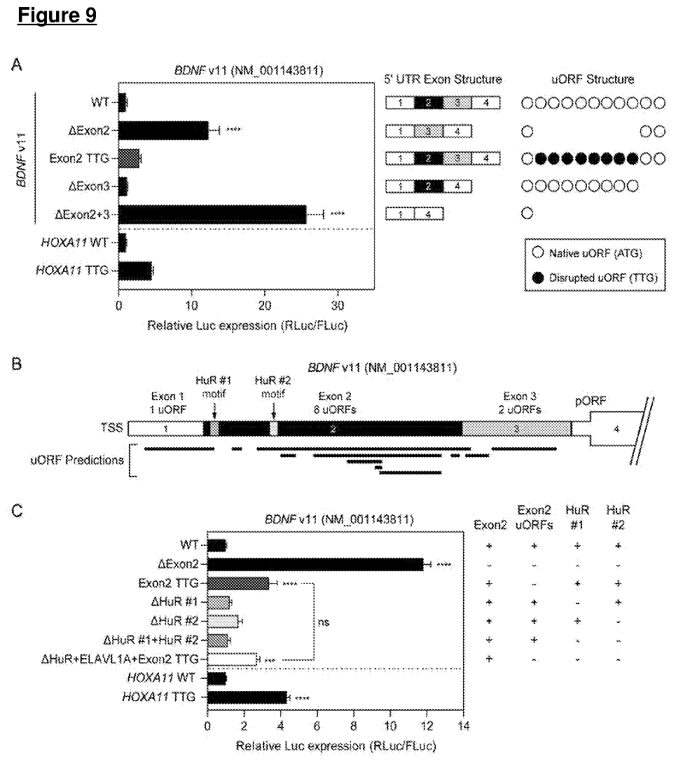

Based on these findings BDNF v11 was selected for further analysis. It was hypothesised that the upregulation observed when exon 2 was deleted might be a consequence of the exclusion of 8 uORF sequences from the resulting ‘exon skipped’ mRNA. To this end, constructs were tested in which all 8 exon 2 uORFs were disrupted by mutating their start codon ATG trinucleotides to TTG. Disruption of the exon 2 uORF resulted in ˜3 fold upregulation in reporter gene expression (FIG. 9A), consistent with the relieving of uORF-mediated repression. Interestingly, this upregulation was only a fraction of that observed when exon 2 was deleted in its entirety, suggesting that while uORFs contribute to pORF repression, they cannot account for all of the repressive activity.

Analysis of the sequence of BDNF v11 exon 2 using BRIO (BEAM RNA Interaction mOtifs) (reference 30) identified two RNA binding motifs of interest (HuR #1 and HuR #2) (FIG. 9B). However, deletion of these motifs, both individually and together, had no significant effect on pORF reporter expression (FIG. 9C), suggesting that they are not responsible for the translation repressing activity contained within exon 2.

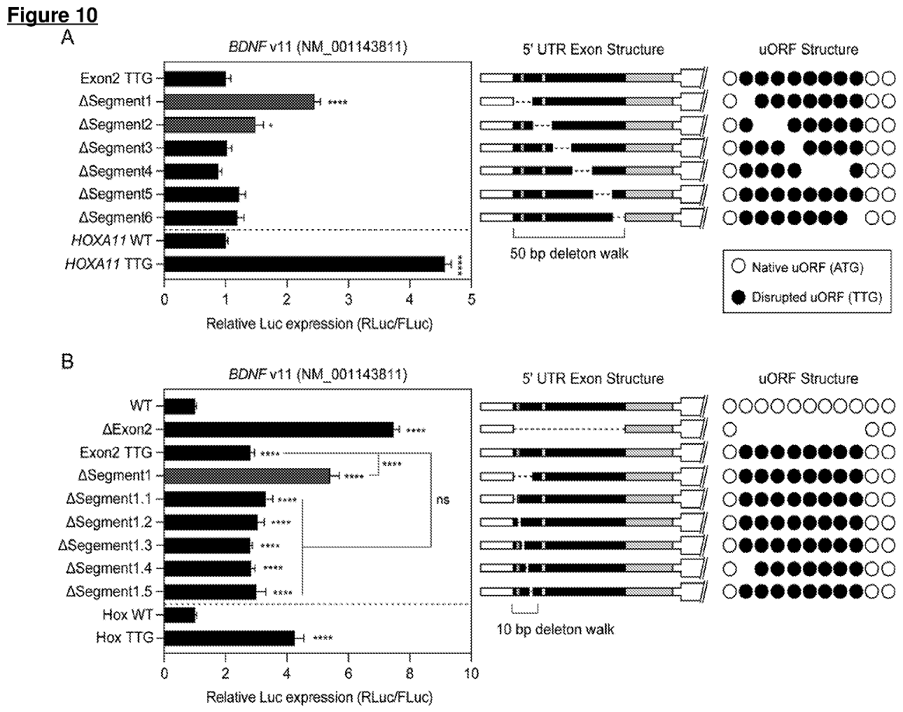

Next, deletion walks were performed to identify sequences in the BDNF v11 exon 2 that could account for its translation repressive activity. Mutant constructs were generated in which 50 bp regions of exon 2 were sequentially deleted. For the purpose of this experiment, all uORFs within exon 2 were disrupted, such that non-uORF repressive elements could be identified. Deletion of the first 50 bp (ΔSegment 1) resulted in a pronounced 3-fold upregulation in reporter activity relative to the control construct where all the uORFs in exon 2 were disputed (FIG. 10A). These data suggest that the combination of uORF-mediated repression, together with a further motif contained within these first 50 bp accounts for the majority of the repressive activity of this exon. A second series of deletion mutants were generated whereby 10 bp regions were deleted across the 50 bp region of interest at the start of exon 2. No upregulation activity was observed for any of these constructs (FIG. 10B).

Together these data show that complex signals contained within exons may be excluded from the mature mRNA transcript for the purposes of targeted gene upregulation. These signals include, but are not limited to, uORFs. These signals may include RNA binding motifs and/or RNA structural features.

TABLE 3
uORF-containing genes and associated diseases

| | NCBI | |
|---|---|---|
| Gene | Gene ID | Associated diseases |
| ABCA1 | 19 | Cardiovascular, Dry AMD, dyslipidemia, and |
| | | atherosclerosis |
| ABCB11 | 8647 | Cholestasis, primary sclerosing cholangitis |
| | | and biliary cirrhosis |
| ABCC2 | 1244 | Dubin-Johnson syndrome (but overexpressed |
| | | in cancer) |
| ABCG5 | 64240 | Cholestasis, primary sclerosing cholangitis |
| | | and biliary cirrhosis |
| ADAM10 | 102 | Alzheimer's Disease |
| ALB | 213 | liver disease, nephrotic syndrome, renal |
| | | disease, and analbuminemia |
| ANK1 | 286 | Hereditary spherocytosis |
| APOE | 348 | Cancer, melanoma, pulmonary hypertension, |
| | | dyslipidemia, atherosclerosis, Alzheimer |
| | | disease, Lipoprotein glomerulopathy, and |
| | | Sea-blue histiocyte disease |
| ATP2A2 | 488 | cardiac diseases, congenital heart disease, |
| | | aortic aneurysms, aortic dissections, arrhythmia, |
| | | cardiomyopathy, congestive heart failure, |
| | | Darier White disease, muscular dystrophy, |
| | | and Acrokeratosis verruciformis |
| ATP7B | 540 | wilson disease, and menkes disease. |
| ATRX | 546 | alpha-thalassemia myelodysplasia syndrome, |
| | | somatic, and mental retardation-hypotonic |
| | | facies syndrome, x-linked. |
| ATXN1 | 6310 | Spinocerebellar ataxia-1 |
| ATXNIL | 342371 | Spinocerebellar ataxia-1 |
| BAX | 581 | Cancer |
| BCL2L11 | 10018 | Cancer, e.g. human T-cell acute lymphoblastic |
| | | leukemia and lymphoma |
| BDNF | 627 | neurodegeneration diseases, amyotrophic lateral |
| | | sclerosis, Alzheimer's Disease, Huntington's |
| | | disease (HD), or Parkinson's Disease (PD) |
| BLM | 641 | bloom syndrome, and rothmund-thomson syndrome |
| BRCA1 | 672 | Cancer, e.g. breast cancer, pancreatic cancer |
| C/EBPa | 1050 | B-cell maligancy (B-ALL, DLBCL), AML |
| CA2 | 760 | autoimmune retinopathy, and multifocal fibrosclerosis |
| CASP8 | 841 | CASP8 deficiency, breast cancer, HCC, lung cancer |
| CCBE1 | 147372 | hennekam syndrome, and immune hydrops fetalis |
| CD36 | 948 | platelet glycoprotein IV deficiency, coronary |
| | | heart disease, CHDS7 |
| CD3D | 915 | severe combined immune deficiency, autosomal |
| | | recessive, T cell-negative, b cell-positive, nk |
| | | cell-positive, cd3d-related, and immunodeficiency 19 |
| CDKN1B | 1027 | cancer, multiple endocrine neoplasia |
| CDKN2A | 1029 | cancer, melanoma |
| CEP290 | 80184 | Leber's congenital amaurosis (LCA), Bardet-Biedl |
| | | syndrome (BBS), Joubert syndrome, Meckel syndrome, |
| | | Sior-Loken syndrome |
| CFH | 3075 | C3 glomerulopathy, AMD, PNH, RA etc |
| CFTR | 1080 | Cystic fibrosis, Disseminated bronchiectasis, |
| | | congenital bilateral absence of vas deferens (CBAVD) |
| CHRNA4 | 1137 | nicotine addiction |
| CHRNA5 | 1138 | nicotine addiction |
| CNTF | 1270 | Multiple Sclerosis |
| CNTFR | 1271 | Multiple Sclerosis |
| COL1A1 | 1277 | Osteogenesis Imperfecta Type I |
| CR1 | 1378 | Alzheimer's Disease |
| CSPP1 | 79848 | joubert syndrome 21, and joubert syndrome with |
| | | jeune asphyxiating thoracic dystrophy. |
| CTNND2 | 1501 | Cri-du-chat syndrome |
| CTNS | 1497 | intermediate cystinosis, and cystinosis, atypical |
| | | nephropathic |
| CYP1B1 | 1545 | Glaucoma, Peters anomaly |
| DBT | 1629 | maple syrup urine disease type 2, and maple syrup |
| | | urine disease type la |
| DCAF17 | 80067 | sakati syndrome, and hypogonadism, alopecia, diabetes |
| | | mellitus, mental retardation, and extrapyramidal syndrome |
| DNASE1 | 1773 | cystic fibrosis, acute bronchitis |
| DDIT3 | 1649 | Myxoid liposarcoma |
| DICER1 | 23405 | DICER1 syndrome, pleuropulmonary blastoma, cystic nephroma, |
| | | Sertoli Leydig tumors, multinodular goiter, cancer |
| DRD3 | 1814 | mood disorders |
| EED | 8726 | HIV-1 |
| EFNB1 | 1947 | CFNS |
| EPO | 2056 | erythropoiesis and anemia |
| ESR1 | 2099 | inhibits ERBB1, breast cancer |
| ETHE1 | 23474 | ethylmalonic encephalopathy |
| EZH2 | 2146 | weaver syndrome, ezh2-related overgrowth, lymphomas and leukemias |
| F8 (and F2, | 2147, 2152, | Hemophilia, bleeding |
| 3, 5, 7, | 2153, 2155, | |
| 11, 13) | 2157, 2160 | |
| FAP | 2191 | glomuvenous malformations |
| FMR1 | 2332 | Fragile X syndrome and premature ovarian failure |
| FNDC5 | 252995 | Obesity, Type 2 Diabetes |
| FXN | 2395 | Friedreich's ataxia |
| GALNS | 2588 | mucopolysaccharidosis iv, and kniest dysplasia |
| GATA3 | 2625 | Cancer |
| GBA | 2629 | Synucleinopathies, Gaucher's disease |
| GCH1 | 2643 | CNS disease, dopa-responsive dystonia, hyperphenylalaninemia, |
| | | and atypical severe phenylketonuria |
| GCK | 2645 | Obesity, Type 2 Diabetes, and Hyper |
| GH2 | 2689 | idiopathic short stature, growth delay |
| GRN | 2896 | autoimmune, inflammatory, dementia, FTD, cancer, e.g. hepatic cancer |
| HBB | 3043 | thalassemia, sickle cell disease, and anemia |
| HBD | 3045 | thalassemia, sickle cell disease, and anemia |
| HBE1 | 3046 | thalassemia, sickle cell disease, and anemia |
| HBG1 | 3047 | Anemia (e.g. Fanconi's anemia), thalassemia (e.g.beta-thalassemia etc.), |
| | | sickle cell disease, leukemia, cellular dyscrasia, dyserythropoiesis, |
| | | anisocytosis and poikilocytosis. |
| HBG2 | 3048 | thalassemia, sickle cell disease, and anemia |
| HCRT | 3060 | Narcolepsy/Excessive Daytime Sleepiness |
| HGF | 3082 | Ischemic disease, restenosis after percutaneous transluminal coronary |
| | | angioplasty (PTCA), arteriosclerosis, insufficiency of peripheral |
| | | circulation, myocardial infarction, myocardia, peripheral angiostenosis, |
| | | cardiac insufficiency, nerve degeneration, neuropathy, neurotoxin induced |
| | | lesions, injury of nerve cell, lesions of nerve cell by infection, epilepsy, |
| | | head trauma, dementia, cerebral stroke, cerebral infarction, amyotrophic |
| | | lateral sclerosis, Parkinson's disease, Alzheimer's disease, |
| | | cancer, tumor, liver cirrhosis, Nonalcoholic fatty liver disease, renal |
| | | fibrosis, rhabdomyolysis, pulmonary fibrosis, blood coagulopathy, adenosine |
| | | deaminase deficiency, Chronic Ulcerative Colitis, Crohn's Disease, |
| | | necrotizing enterocolitis, severe acute gastroenteritis, chronic |
| | | gastroenteritis, cholera, chronic infections of the bowel, AIDS, pustulous |
| | | fibrosis, fibrosis, osteoporosis, Arterial sclerosis, chronic glomerulonephritis, |
| | | cutis keloid formation, progressive systemic sclerosis (PSS), liver fibrosis, |
| | | pulmonary fibrosis, cystic fibrosis, chronic graft versus host disease, |
| | | scleroderma (local and systemic), Peyronie's disease, penis fibrosis, |
| | | inner accretion after surgery, myelofibrosis, idiopathic retroperitoneal |
| | | fibrosis, hemophilia, decubitus ulcer, scar, atopic dermatitis, or skin |
| | | damage following a skin graft |
| HNF4a | 3172 | HCC, fibrosis |
| HR | 55806 | atrichia with papular lesions, and hypotrichosis-4 |
| HSD17B4 | 3295 | D-bifunctional protein deficiency |
| IDO1 | 3620 | autoimmune and inflammatory diseases |
| IFNE and other | 338376, others | Cancer, HBV, and other virus infection |
| interferon genes | | |
| IFRD1 | 3475 | Cystic fibrosis, Chronic obstructive pulmonary disease (COPD), inflammation, |
| | | lung cancer, sensory/motor neuropathy, a neuronal injury |
| IGF1 | 3479 | CNS diseases, metabolic disease, delayed growth, cancer |
| IGF1R | 3480 | Insulin-like growth factor I resistance |
| IGF2 | 3481 | Russell-Silver syndrome |
| IGF2BP2 | 10644 | Type 2 diabetes, insulin resistance susceptibility |
| IGFBP3 | 3486 | growth delay |
| IGHMBP2 | 3508 | progressive multifocal leukoencephalopathy, and spinal |
| | | muscular atrophy with respiratory distress |
| IL6 | 3569 | infectious disease, vaccination, and cancer |
| INS | 3630 | Diabetes or related disorders thereof, an insulin resistant non |
| | | diabetic state, obesity, impaired glucose tolerance (IGT), Metabolic |
| | | Syndrome, MODY syndrome, Polycystic Ovary Syndrome, cancer, |
| | | inflammation, hirsuitism, and hypertension. |
| IQGAP1 | 8826 | Cancer, obesity, diabetes, multiple sclerosis, neoplastic transformation, |
| | | inflammation, Nonsmall cell lung carcinoma (NSCLCs), hypercholesterolemia, |
| | | liposarcoma, gastric cancer, immunodeficiency, glomerulonephritis, venous |
| | | thrombosis, glioma |
| IQGAP2 | 10788 | Obesity, diabetes, multiple sclerosis, neoplastic transformation, |
| | | inflammation, Nonsmall cell lung carcinoma (NSCLCs), hypercholesterolemia, |
| | | liposarcoma, gastric cancer, immunodeficiency, glomerulonephritis, |
| | | venous thrombosis, glioma |
| IRF6 | 3664 | van der Woude syndrome |
| IRS2 | 8660 | Alzheimer's disease, Parkinson's disease, amyotrophic lateral |
| | | sclerosis, insulin resistance, diabetes, Polycystic Ovary Syndrome, |
| | | atherosclerosis, cancer |
| ITGA7 | 3679 | muscular dystrophy, congenital, due to itga7 deficiency, and congenital |
| | | muscular dystrophy due to integrin alpha-7 deficiency |
| JAG1 | 182 | Alagille syndrome |
| KCNJ11 | 3767 | Congenital hyperinsulinism, hyperinsulinemic hypoglycemia, 2 |
| KCNMA1 | 3778 | vascular disease, kidney disease, Obesity, Type 2 Diabetes, inflammatory |
| | | disease, autoimmune disease, and cancer, e.g. kidney, lung, or ovarian cancer |
| KCNMB1 | 3779 | vascular disease, kidney disease, Obesity, Type 2 Diabetes, inflammatory |
| | | disease, autoimmune disease, and cancer, e.g. kidney, lung, or ovaria cancer |
| KCNMB2 | 10242 | vascular disease, kidney disease, Obesity, Type 2 Diabetes, inflammatory |
| | | disease, autoimmune disease, and cancer, e.g. kidney, lung, or ovarian cancer |
| KCNMB3 | 27094 | vascular disease, kidney disease, Obesity, Type 2 Diabetes, inflammatory |
| | | disease, autoimmune disease, and cancer, e.g. kidney, lung, or ovarian cancer |
| KCNQ3 | 3786 | kcnq3-related benign familial neonatal epilepsy, and seizures, |
| | | benign neonatal, type 2 |
| KLF4 | 9314 | thalassemia, sickle cell disease, and anemia |
| KMT2D | 8085 | Kabuki Syndrome |
| LDLR | 3949 | dyslipidemias, atherosclerosis, and hypercholesterolemia, cardiovascular |
| | | disease |
| LRP1 | 4035 | Cancer, melanoma |
| LRP5, | 4041 | exudative vitreoretinopathy 4, and hyperostosis, endosteal |
| LRP8 | 7804 | Cancer, melanoma |
| LRPPRC | 10128 | Leigh syndrome French-Canadian type, Cytochrome c oxidase deficiency |
| MBTPS1 | 8720 | Colitis, obesity, diabetes, hypercholesterolemia, dyslipidemia, |
| | | Crimean-Congo hemorrhagic fever, chondrodysplasia |
| MECP2 | 4204 | Rett Syndrome, MECP2-related severe neonatal encephalopathy, Angelman |
| | | syndrome, and PPM-X syndrome |
| MSRA | 4482 | cancer, macular degeneration, eye aging, cataract |
| MSX2 | 4488 | tooth agenesis (dentin dysplasia), developmental disorders e.g. |
| | | Craniosynostosis and Parietal foramina |
| MTR | 4548 | Homocystinuria |
| MUTYH | 4595 | Familial adenomatous polyposis |
| MYCN | 4613 | Feingold syndrome |
| MYF6 | 4618 | Centronuclear Myopathy 3 |
| NAMPT | 10135 | cancer, cytopenia of the myeloid or lymphoid lineage, neutropenia, |
| | | leukaemia, acute myeloid leukaemia (AML), atherosclerosis, inflammatory |
| | | bowel disease, Crohn's disease, ulcerative colitis, psoriasis, |
| | | arthritis, chronic ulcer, ischemic stroke, myocardial infarction, |
| | | angina and vascular dementia, inflammation, nonalcoholic fatty liver disease |
| NANOG | 79923 | diabetes, osteoarthritis, rheumatoid arthritis, cancer, Duchenne muscular |
| | | dystrophy, Parkinson's, Alzheimer's, Gaucher disease, type I diabetes, |
| | | spinal cord injury, burns (tissue regeneration) |
| NEU4 | 129807 | cancer, diabetes, Tay Sachs disease, inflammatory bowel disease, Crohn's |
| | | disease, ulcerative colitis, psoriasis, arthritis, inflammation, insulin |
| | | resistance syndrome, hyperlipidemia, fatty liver disease, cachexia, obesity, |
| | | atherosclerosis, arteriosclerosis, elevated blood pressure, viral infection |
| NF1 | 4763 | neurofibromatosis and cancer, e.g., neurofibrosarcoma, malignant peripheral |
| | | nerve sheath tumors, and myelomonocytic leukemia |
| NKX2-3, −5, −8 | 159296, 1482, 26257 | cancer, e.g., lung cancer |
| NOD2 | 64127 | Crohn disease |
| NR5A1 | 2516 | nr5al-related 46, xy dsd and 46, xy cgd, and adrenocortical insufficiency, |
| | | without ovarian defect |
| NRF1 | 4899 | Alzheimer's disease, Parkinson's disease, amyotrophic lateral sclerosis, |
| | | insulin resistance, diabetes, hepatic tumor, non-small cell bronchopulmonary |
| | | cancer, mitochondrial disease |
| NSD1 | 64324 | Sotos syndrome (cerebral gigantism)autosomal dominant disorder. The cause |
| | | is haploinsifficiency of the NSD1 gene |
| PAH | 5053 | Phenylketonuria (PKU) |
| PARK2 | 5071 | Parkinson's |
| PKD1 | 5310 | Polycystic kidney disease |
| PLAT | 5327 | ischemic stroke |
| PON1, 2 | 5444, 5445 | diabetes, obesity, hypercholesterolemia, high blood pressure, atherosclerosis, |
| | | coronary heart disease, autism/autism spectrum disorder, epilepsy, cancer, |
| | | inflammation, stroke, trauma, a renal disease, rheumatoid arthritis, Fish-Eye |
| | | disease, purpura, Polycystic Ovary Syndrome, hyperthyroidism, a hepatic |
| | | diseases, vascular dementia, an infectious disease |
| PPARD | 5467 | Metabolic disease |
| PRKAR1A | 5573 | Carney complex |
| PRPF31 | 26121 | adRP |
| PTEN | 5728 | cancer |
| PYCR1 | 5831 | cystic fibrosis, myocardial fibrosis, myelofibrosis, hepatic fibrosis, |
| | | interstitial lung fibrosis, neoplastic fibrosis, pancreatic fibrosis, |
| | | pulmonary fibrosis, subepidermal fibrosis, panmural fibrosis of the |
| | | bladder, proliferative fibrosis, replacement fibrosis, retroperitoneal |
| | | fibrosis and root sleeve fibrosis, osteogenesis imperfecta, Ehlers-Danlos |
| | | syndrome, chondrodysplasias, Marfan syndrome, Alport syndrome, familial |
| | | aortic aneurysm, achondroplasia, mucopolysaccharidoses, osteoporosis, |
| | | osteopetrosis, Paget's disease, rickets, osteomalacia, hyperparathyroidism, |
| | | renal osteodystrophy, osteonecrosis, osteomyelitis, osteoma, osteoid osteoma, |
| | | osteoblastoma, osteosarcoma, osteochondroma, chondroma, chondroblastoma, |
| | | chondromyxoid fibroma, chondrosarcoma, fibrous cortical defect, nonossifying |
| | | fibroma, fibrous dysplasia, fibrosarcoma, malignant fibrous histiocytoma, |
| | | Ewing's sarcoma |
| RBI, RBL1, | 5925 | cancer, e.g. bladder cancer, osteosarcoma, retinoblastoma, small cell lung |
| RBL2 | 5933, 5934 | cancer |
| RBBP4 | 5928 | intermediate charcot-marie-tooth neuropathy, retinoblastoma, Alzheimer's |
| RNASEH1 | 246243 | leishmaniasis, a disease or disorder associated with mitochondrial dysfunction, |
| | | cancer, Aicardi-Goutieres syndrome, AIDS |
| ROR2 | 4920 | brachydactyly, type bl, and brachydactyly type b |
| RPS14 | 6208 | 5q syndrome (myelodysplastic syndrome) |
| RPS19 | 6223 | Diamond-Blackfan Anemia |
| SCN1A | 6323 | convulsion, pain, paralysis, hyperkalemic periodic paralysis, paramyotonia |
| | | congenita, potassium-aggravated myotonia, long Q-T syndrome 3, motor |
| | | endplate disease, ataxia, colitis, ileitis, inflammatory bowel syndrome, |
| | | hypertension, congestive heart failure, benign prostrate hyperplasia, impotence, |
| | | muscular dystrophy, multiple sclerosis, epilepsy, autism, migraine, severe |
| | | myoclonic epilepsy of infancy (SMEI or Dravet's syndrome) |
| SCN2A | 6326 | epileptic encephalopathy, early infantile, 11, and benign familial |
| | | neonatal-infantile seizures |
| SERPINF1 | 5176 | cancer, choroidal neovascularization, cardiovascular disease, diabetes, |
| | | and osteogenesis imperfecta |
| SERPING1 | 710 | Hereditary Angioedema |
| SHBG | 6462 | Disorders of mood and affect, a memory dysfunction disease or disorder, an |
| | | amnestic disease or disorder, a motor and tic disorder, substance abuse |
| | | disease or disorder, a psychotic disease or disorder, an anxiety disease |
| | | or disorder, schizophrenia, schizofreniform disorder, schizoaffective |
| | | disorder, and delusional disorder, panic disorder, phobias, an obsessive- |
| | | compulsive disorder, posttraumatic stress disorder, infertility, hirsutism, |
| | | Tourette's disorder, Asperger syndrome, hypothyroidism, fibromyalgia, chronic |
| | | fatigue syndrome, hypothalamic-pituitary axis dysregulation, chronic sleep |
| | | deprivation, alopecia, prostate cancer, breast cancer, polycystic ovary |
| | | syndrome, osteoporosis, hyperinsulinemia, glucose intolerance, insulin |
| | | resistance, diabetes |
| SIRT1 | 23411 | cancer, Alzheimer's Disease (AD), Huntington's disease, Parkinson's |
| | | disease, Amyotrophic Lateral Sclerosis (ALS), Multiple Sclerosis, Duchene |
| | | muscular dystrophy, skeletal muscle atrophy, Becker's dystrophy, myotonic |
| | | dystrophy, insulin resistance, diabetes, obesity, Hypercholesterolemia, dyslipidemia |
| | | hyperlipidemia, sensory neuropathy, autonomic neuropathy, motor neuropathy, |
| | | retinopathy, hepatitis, fatty liver disease, age-related macular degeneration, |
| | | osteoporosis, leukemia, bone resorption, dementia, Bell's Palsy, atherosclerosis, |
| | | cardiac dysrhymias, chronic congestive heart failure, ischemic stroke, coronary |
| | | artery disease, cardiac muscle disease, chronic renal failure, ulceration, cataract, |
| | | presbiopia, glomerulonephritis, Guillan Barre syndrome, hemorrhagic stroke, rheumatoid |
| | | arthritis, inflammatory bowel disease, SLE, Crohn's disease, osteoarthritis, |
| | | Chronic Obstructive Pulmonary Disease (COPD), pneumonia, urinary incontinence, |
| | | mitochondrial myopathy, encephalopathy, Leber's disease, Leigh encephalopathia, |
| | | Pearson's disease, lactic acidosis, mitochondrial encephalopathy, lactic |
| | | acidosis and stroke like symptoms (MELAS), inflammation |
| SLC1A2 | 6506 | ALS |
| SMAD7 | 4092 | Acute kidney injury (anti-TGFb), colorectal cancer |
| SMCHD1 | 23347 | FSHD |
| SMN1, SMN2 | 6606, 6607 | Spinal muscular atrophy |
| 5NX27 | 81609 | Downs' Syndrome |
| SPINK1 | 6690 | Pancreatitis |
| SRB1 | 949 | Cardiovascular disease |
| SRY | 6736 | Gonadal dysgenesis |
| ST7, ST7L | 7982, 54879 | cancer, e.g. myeloid cancer, head and neck squamous cell carcinomas, breast |
| | | cancer, colon carcinoma, and prostate cancer |
| STAT3 | 6774 | tissue regeneration and Hyper-IgE recurrent infection syndrome |
| TFE3 | 7030 | Diabetes, obesity, impaired glucose tolerance (IGT) and Metabolic Syndrome, |
| | | Polycystic Ovary Syndrome, atherosclerosis, cancer, Alzheimer's disease, |
| | | Parkinson's disease, amyotrophic lateral sclerosis, diabetic retinopathy, |
| | | diabetic neuropathy, diabetic amyotrophy, diabetic nephropathy, diabetic |
| | | cardiomyopathy, angina, myocardial infarction, stroke, a peripheral vascular disease |
| TFEB | 7942 | Lysosomal storage diseases |
| TGFB3 | 7043 | Rienhoff syndrome |
| THPO | 7066 | Myelosuppressive chemo, Bleeding disorders |
| TP63 | 8626 | cancer, tumor, Corneal dystrophy, premature menopause, alopecia, cctrodaclyly- |
| | | ectodermal dysplasia-cleft syndrome, Hay-Wells syndrome, limb mammary syndrome, |
| | | acro-dermato-ungual-lacrimal-tooth syndrome, nonsyndromic split-hand/foot |
| | | malformation, isolated cleft lip/palate, Rapp Hodgkin syndrome |
| TP73 | 7161 | Cancer |
| UCP2 | 7351 | cancer, obesity, cachexia, anorexia nervosa, bulimia nervosa, diabetes, |
| | | hyperinsulinemia, glucose intolerance, atherosclerosis, inflammation |
| USP9Y/SP3 | 8287 | Y chromosome infertility |
| UTRN | 7402 | muscular dystrophies, Duchenne muscular dystrophy (DMD), Becker Muscular |
| | | Dystrophy (BMD), and myotonic dystrophy |
| VEGFA | 7422 | diabetes, coronary artery disease, congestive heart failure, and peripheral |
| | | vascular disease, cancer, infectious diseases, rheumatoid arthritis, DiGeorge |
| | | syndrome, HHT, cavernous hemangioma, atherosclerosis, transplant ateriopathy, |
| | | obesity, psoriasis, warts, allergic dermatitis, scar keloids, pyogenic |
| | | granulomas, blistering disease, Kaposi sarcoma, persistent hyperplastic |
| | | vitreous syndrome, Autosomal dominant polycystic kidney disease (ADPKD), |
| | | diabetic retinopathy, retinopathy of prematurity, macular degeneration, |
| | | choroidal neovascularization, primary pulmonary hypertension, asthma, nasal |
| | | polyps, inflammatory bowel disease, Alzheimer's disease, Parkinson's |
| | | disease, amyotrophic lateral sclerosis, periodontal disease, ascites, |
| | | peritoneal adhesions, endometriosis, uterine bleeding, ovarian cysts, ovarian |
| | | hyperstimulation, arthritis, synovitis, osteomyelitis, and/or osteophyte |
| | | formation, ulceration, verruca vulgaris, angiofibroma of tuberous sclerosis, |
| | | pot-wine stains, Sturge Weber syndrome, Kippel-Trenaunay-Weber syndrome, |
| | | Osler-Weber-Rendu syndrome |

TABLE 4
Genes with mutations or SNPs that create uORFs and associated diseases.

| | NCBI | |
|---|---|---|
| Gene | Gene ID | Associated disease(s) |
| ATP7B | 540 | wilson disease, and menkes disease |
| ATRX | 546 | alpha-thalassemia myelodysplasia syndrome, somatic, |
| | | and mental retardation-hypotonic facies syndrome, x-linked |
| BLM | 641 | bloom syndrome, and rothmund-thomson syndrome |
| BRCA1 | 672 | primary peritoneal carcinoma, and hereditary site-specific |
| | | ovarian cancer syndrome |
| CA2 | 760 | autoimmune retinopathy, and multifocal fibrosclerosis |
| CCBE1 | 147372 | hennekam syndrome, and immune hydrops fetalis |
| CD3D | 915 | severe combined immune deficiency, autosomal recessive, |
| | | t cell-negative, b cell-positive, nk cell-positive, cd3d- |
| | | related, and immunodeficiency 19 |
| CD4 | 920 | okt4 epitope deficiency, and lymphatic system disease |
| CDKN2A | 1029 | Melanoma predisposition, Melanoma |
| CFL2 | 1073 | cfl2-related nemaline myopathy, and nemaline myopathy 7, |
| | | autosomal recessive |
| CFTR | 1080 | Cystic fibrosis, Disseminated bronchiectasis |
| CSPP1 | 79848 | joubert syndrome 21, and joubert syndrome with jeune |
| | | asphyxiating thoracic dystrophy |
| CTNS | 1497 | intermediate cystinosis, and cystinosis, atypical nephropathic |
| DBF | 1629 | maple syrup urine disease type 2, and maple syrup urine |
| | | disease type la |
| DCAF17 | 80067 | sakati syndrome, and hypogonadism, alopecia, diabetes mellitus, |
| | | mental retardation, and extrapyramidal syndrome |
| DCLRE1C | 64421 | severe combined immunodeficiency, athabascan type, and |
| | | artemis deficiency |
| DFNB31 | 25861 | deafness, autosomal recessive 31, and dfnb31 nonsyndromic |
| | | hearing loss and deafness |
| DLG4 | 1742 | schizophrenia |
| DMD | 1756 | duchenne muscular dystrophy, and becker muscular dystrophy |
| DNASE1 | 1773 | cystic fibrosis, acute bronchitis |
| ETHE1 | 23474 | ethylmalonic encephalopathy |
| GALNS | 2588 | mucopolysaccharidosis iv, and kniest dysplasia |
| GCH1 | 2643 | Levodopa responsive dystonia |
| HAMP | 57817 | Juvenile hemochromatosis, thalassemia |
| HBB | 3043 | Beta-Thalassemia |
| HMBS | 3145 | histrionic personality disorder, and acute porphyria |
| HR | 55806 | atrichia with papular lesions, and hypotrichosis 4 |
| IGHMBP2 | 3508 | progressive multifocal leukoencephalopathy, and spinal |
| | | muscular atrophy with respiratory distress 1 |
| IRF6 | 3664 | Van der Woude syndrome |
| ITGA7 | 3679 | muscular dystrophy, congenital, due to itga7 deficiency, |
| | | and congenital muscular dystrophy due to integrin alpha-7 deficiency |
| ITGB2 | 3689 | leukocyte adhesion deficiency type 1, and leukocyte adhesion deficiency. |
| KCNJ11 | 3767 | Congenital hyperinsulinism, hyperinsulinemic hypoglycemia, 2 |
| KCNQ3 | 3786 | kcnq3-related benign familial neonatal epilepsy, and seizures, |
| | | benign neonatal, type 2 |
| LDLR | 3949 | Cardiovascular disease, Familial hypercholesterolemia |
| LRP5, LRP5L | 4041, 91355 | exudative vitreoretinopathy 4, and hyperostosis, endosteal |
| MECP2 | 4204 | autism susceptibility, x-linked 3, and bruxism. |
| MLH1 | 4292 | mlhl-related lynch syndrome, and solitary rectal ulcer syndrome. |
| MSH6 | 2956 | msh6-related lynch syndrome, colorectal cancer, hereditary |
| | | nonpolyposis, type 5 |
| MUTYH | 4595 | adenomas, multiple colorectal, stomach cancer. |
| NR5A1 | 2516 | nr5al-related 46,xy dsd and 46,xy cgd, and adrenocortical |
| | | insufficiency, without ovarian defect |
| PALB2 | 79728 | fanconi anemia, complementation group n, and pancreatic cancer |
| | | susceptibility 3. |
| PANK2 | 80025 | classic pantothenate kinase-associated neurodegeneration, |
| | | and harp syndrome. |
| PEX7 | 5191 | peroxisome biogenesis disorder 9b, and rhizomelic chondrodysplasia |
| | | punctata. |
| PHYH | 5264 | phyh-related refsum disease, and refsum disease. |
| PIK3R5 | 23533 | ataxia-oculomotor apraxia 3, and spinocerebellar ataxia autosomal |
| | | recessive 1 |
| POMC | 5443 | Proopiomelanocortin deficiency |
| POMT1 | 10585 | pomtl-related muscle diseases, and walker-warburg syndrome. |
| ROR2 | 4920 | brachydactyly, type bl, and brachydactyly type b |
| SCN2A | 6326 | epileptic encephalopathy, early infantile, 11, and benign |
| | | familial neonatal-infantile seizures |
| SGCA | 6442 | sarcoglycanopathies, and limb-girdle muscular dystrophy, type 2d. |
| SGCD | 6444 | limb-girdle muscular dystrophy type 2f, and delta-sarcoglycanopathy. |
| SLC16A1 | 6566 | erythrocyte lactate transporter defect, and exercise-induced |
| | | hyperinsulinemic hypoglycemia. |
| SLC19A3 | 80704 | basal ganglia disease, and biotin-responsive basal ganglia disease. |
| SLC2A2 | 6514 | fanconi bickel syndrome, fanconi syndrome |
| SLC7A9 | 11136 | stinuria, and aminoaciduria |
| SPINK1 | 6690 | Hereditary pancreatitis |
| SRY | 6736 | Gonadal dysgenesis |
| STIL | 6491 | primary autosomal recessive microcephaly type 7, and ideomotor apraxia. |
| TK2 | 7084 | mitochondrial dna depletion syndrome, myopathic form, and tk2-related |
| | | mitochondrial dna depletion syndrome, myopathic form. |
| TMPRSS3 | 64699 | deafness, autosomal recessive 8/10, and dfnb 8/10 nonsyndromic hearing |
| | | loss and deafness. |
| TP53 | 7157 | hepatocellular carcinoma, and osteosarcoma |
| TPI1 | 7167 | hemolytic anemia due to triosephosphate isomerase deficiency, and |
| | | triose phosphate-isomerase deficiency. |
| TPM3 | 7170 | nemaline myopathy, and nemaline myopathy 1 |
| TRMU | 55687 | liver failure acute infantile, and melas, mt-th-related. |
| TSEN54 | 283989 | tsen54-related pontocerebellar hypoplasia, and pontocerebellar |
| | | hypoplasia type 4 |
| ZEB1 | 6935 | comeal dystrophy, posterior polymorphous, 3, and corneal dystrophy, |
| | | fuchs endothelial corneal dystrophy-6. |

### REFERENCES

1 Wang et al., Molecular Systems Biology, 15:e8503 (2019).
2 Sousa and Farkas, PLoS Genet 14(12): e1007764 (2018).
3 Matsui et al., FEBS let. 581: 4184/4188 (2007).
4 Morris and Gabelle, Mol. Cell. Biol. 20, 8635-8642 (2000).
5 Aartsma-Rus and Ommen, RNA 13: 1609-1624 (2007).
6 Fairbrother et al., Nucleci Acids Res. 2004, 32 (Web Server issue):W187-190.
7 Cartgegni et al., Nucleic Acids Res. 2003 31(13):3568-71.
8 Crooke et al., Nat Biotechnol. 2017 March; 35(3):230-237.
9 Roberts et al., Nat Rev Drug Discov. 2020 October; 19(10):673-694.
10 Crooke et al., Nat Biotechnol. 2017 March; 35(3):230-237.
11 Geary et al., Adv Drug Deliv Rev. 2015 Jun. 29; 87:46-51.
12 Summerton, Biochim Biophys Acta. 1999 Dec. 10; 1489(1):141-58.
13 U.S. Pat. No. 5,142,047.
14 U.S. Pat. No. 5,185,444.
15 WO2009099942.
16 EP1579015.
17 Griffey et al., J. Med. Chem. 39(26):5100-5109 (1997))
18 Obika, et al., Tetrahedron Letters 38 (50): 8735 (1997)
19 Koizumi, Current opinion in molecular therapeutics 8 (2): 144-149 (2006)
20 Abramova et al., Indian Journal of Chemistry 48B:1721-1726 (2009)
21 Boisguérin et al., Advanced Drug Delivery Reviews (2015)
22 Lochmann et al., Eu. J. Pharmaceutics and Biopharmaceutics 58 (2004) 237-251
23 Ausubel (ed), Wiley Interscience, New York and the Maniatis Manual produced by Cold Spring Harbor Publishing (1999).
24 Calvo et al., PNAS, 106:7507-7512 (2009).
25 Iacono et al., Gene, 349:97-105 (2005).
26 Yamashita et al., Comptes Rendus Biologies, 326:987-991 (2003).
27 Lee et al., Proc. Natl. Acad. Sci. U.S.A., 109:E2424-2432 (2012).
28 Michel et al., Nucleic Acids Res., 42:D859-864 (2014).
29 Schuller & Green, Nat Rev Mol Cell Biol., 19:526-541 (2018).
30 Guarracino et al., Nucleic Acids Res., 49, W67-W71 (2021).

### Sequence Listing

SEQ ID NOs: 1 to 15 are listed in Table 1.

SEQ ID NOs: 27 to 28 are listed in Table 2.

| SEQ ID NO | Brief description | sequence |
|---|---|---|
| 16 | Kozak consensus sequence | nnnnAUGn |
| 17 | strong Kozak consensus sequence | [a/g]nnAUGg |
| 18 | 5′ splice donor site | [C/A]AGgu[a/g]ag |
| 19 | 3′ splice acceptor site | cagG[G/U] |
| 20 | SRSF1 site | CACACGA |
| 21 | splicing branch-point | cu[a/g]A[c/u] |
| 22 | oligonucleotide sequence targeted to 5′ splice | [N]ₐGARUGGAM[N]_{b} |
| | donor site | |
| 23 | oligonucleotide targeted to 3′ splice acceptor | [N]ₐKGGAC[N]_{b} |
| | site | |
| 24 | oligonucleotide targeted to an exon splicing | [N]ₐUCGUGUG[N]_{b} |
| | enhancer (ESE) sequence | |
| 25 | oligonucleotide sequence targeted to splicing | [N]ₐRUYAG[N]_{b} |
| | branch point | |
| 26 | oligonucleotide sequence targeted to | [N]ₐ[R]_{b}[N]_{c} |
| | polypyrimidine tract | |

N is any nucleotide, or modified or derivative thereof; M is adenosine or cytosine; K is guanosine or uridine; Y is cytosine or uridine; R is adenosine or guanosine; a is 0 to 27; b is 0 to 27; c is 0 to 27.

1. A method for modulating the presence of a regulatory element in a RNA transcript, comprising delivering to a cell a compound targeted to a splicing signal in the RNA transcript to induce splice modulation of one or more exons comprising the regulatory element.

2. The method of claim 1, wherein the regulatory element is a translational regulatory element, such as an upstream open reading frame (uORF).

3. The method of claim 2, wherein the uORF:
(a) is within 100 nucleotides upstream of the primary open reading frame (pORF);
(b) overlaps with the pORF;
(c) comprises the Kozak consensus sequence n[a/g]nnAUGg (SEQ ID NO: 17);
(d) comprises ≤5, ≤4, ≤3, ≤2, 1, or 0 codons between the start codon and the stop codon; and/or
(e) comprises a higher percentage composition of acidic and basic amino acids as compared to aromatic hydrophobic amino acids.

4. The method of claim 2 or 3, wherein the one or more exons comprise ≤10, ≤5 or 1 uORF.

5. The method of any one of claims 2 to 4, wherein the uORF is partially skipped, for example, wherein a portion of the uORF encoding the start codon is skipped.

6. The method of claim 1, wherein the regulatory element is in the 3′ untranslated region (UTR), and the regulatory element is optionally a miRNA binding site or an alternate polyadenylation signal.

7. The method of claim 1, wherein the regulatory element is in a long non-coding RNA transcript, and the regulatory element is optionally a micropeptide-encoding sequence, a RNA-binding domain, or a secondary structure which interacts with proteins.

8. The method of any one of claims 1 to 7, wherein the compound induces exon skipping, optionally wherein the compound induces:
(a) the skipping of a single exon comprising one or more regulatory elements in the RNA transcript; or
(b) the skipping of multiple exons comprising one or more regulatory elements in the RNA transcript.

9. The method of any one of claims 1 to 7, wherein the compound induces inclusion of an exon, optionally wherein the compound induces:
(a) the inclusion of a single exon comprising one or more regulatory elements in the RNA transcript; or
(b) the inclusion of multiple exons comprising one or more regulatory elements in the RNA transcript.

10. The method of any one of the preceding claims, wherein the method comprises delivering to a cell multiple compounds and each compound is targeted to a different target site, and optionally the multiple compounds are conjugated.

11. The method of any one of the preceding claims, wherein the splicing signal comprises:
(a) a 5′ splice donor site;
(b) a 3′ splice acceptor site;
(c) an exon splicing enhancer (ESE) sequence;
(d) a splicing branch point;
(e) a polypyrimidine tract; or
(f) an intronic splicing silencer (ISS) sequence.

12. The method of any one of the preceding claims, wherein the compound is targeted to a target site that is devoid of RNA secondary structures.

13. The method of any one of the preceding claims, wherein the compound is an oligonucleotide.

14. The method of claim 16, wherein the oligonucleotide is an antisense oligonucleotide.

15. The method of claim 16 or claim 17, wherein the oligonucleotide:
(a) is single-stranded;
(b) is 5 to 40 nucleotides in length;
(c) is a modified oligonucleotide; and/or
(d) has ≥50% sequence complementarity to a target site.

16. The method of any one of claims 13 to 15, wherein the oligonucleotide comprises a sequence complementary to a 5′ splice donor site having the sequence [C/A]AGgu[a/g]ag (SEQ ID NO: 18), optionally the oligonucleotide comprises or consists of SEQ ID NO: 22.

17. The method of any one of claims 13 to 15, wherein the oligonucleotide comprises a sequence complementary to a 3′ splice acceptor site having the sequence cagG[G/U] (SEQ ID NO: 19), optionally the oligonucleotide comprises or consists of SEQ ID NO: 23.

18. The method of any one of claims 13 to 15, wherein the oligonucleotide comprises a sequence complementary to an exon splicing enhancer (ESE) sequence (e.g. the SRSF1 site having the sequence CACACGA (SEQ ID NO: 20)), optionally the oligonucleotide comprises or consists of SEQ ID NO: 24.

19. The method of any one of claims 13 to 15, wherein the oligonucleotide comprises a sequence complementary to a splicing branch point having the sequence cu[a/g]A[c/u] (SEQ ID NO: 21), optionally the oligonucleotide comprises or consists of SEQ ID NO: 25.

20. The method of any one of claims 13 to 15, wherein the oligonucleotide comprises a sequence complementary to a polypyrimidine tract, optionally the oligonucleotide comprises or consists of SEQ ID NO: 26.

21. The method of any one of claims 13 to 20, wherein the oligonucleotide has a GC content between 40-60%.

22. The method of any one of claims 13 to 21, wherein the oligonucleotide comprises one or more modifications selected from: at least one modified sugar moiety, at least one modified internucleoside linkage, and/or at least one modified nucleotide.

23. The method of claim 22, wherein the oligonucleotide is a phosphorodiamidate morpholino oligomer (PMO).

24. The method of claim 23, wherein each of the sugar moieties in the oligonucleotide comprises a 2′-O-methoxyethyl modification and each of the internucleoside linkages is a phosphorothioate (i.e. the oligonucleotide is a fully PS-MOE oligonucleotide).

25. The method of any one of the preceding claims, wherein the compound is conjugated to one or more further compounds, such as a nucleic acid molecule, a peptide, or other chemicals.

26. The method of any one of the preceding claims, wherein the RNA transcript is encoded by a uORF-containing gene, such as: ABCA1, ABCB1l, ABCC2, ABCG5, ADAM10, ALB, ANK1, APOE, ATP2A2, ATP7B, ATRX, ATXN1, ATXNIL, BAX, BCL2L11, BDNF (e.g. BDNF v11), BLM, BRCA1, C/EBPα, CA2, CASP8, CCBE1, CD36, CD3D, CDKN1B, CDKN2A, CEP290, CFH, CFTR, CHRNA4, CHRNA5, CNTF, CNTFR, COL1A1, CR1, CSPP1, CTNND2, CTNS, CYP1B1, DBT, DCAF17, DNASE1, DDIT3, DICER1, DRD3, EED, EFNB1, EPO, ESR1, ETHE1, EZH2, F8 (and F2, 3, 5, 7, 11, 13), FAP, FMR1, FNDC5, FXN, GALNS, GATA3, GBA, GCH1, GCK, GH2, GRN, HBB, HBD, HBE1, HBG1, HBG2, HCRT, HGF, HNF4a, HR, HSD17B4, IDO1, IFNE and other interferon genes, IFRD1, IGF1, IGF1R, IGF2, IGF2BP2, IGFBP3, IGHMBP2, IL6, INS, IQGAP1, IQGAP2, IRF6, IRS2, ITGA7, JAG1, KCNJ11, KCNMA1, KCNMB1, KCNMB2, KCNMB3, KCNQ3, KLF4, KMT2D, LDLR, LRP1, LRP5, LRP8, LRPPRC, MBTPS1, MECP2, MSRA, MSX2, MTR, MUTYH, MYCN, MYF6, NAMPT, NANOG, NEU4, NF1, NKX2, NKX3, NKX5, NKX8, NOD2, NR5A, NRF1, NSD1, PAH, PARK2, PKD1, PLAT, PON1, PON2, PPARD, PRKARIA, PRPF31, PTEN, PYCR1, RB1, RBL1, RBL2, RBBP4, RNASEH1, ROR2, RPS14, RPS19, SCNIA, SCN2A, SERPINF1, SERPING1, SHBG, SIRT1, SLC1A2, SMAD7, SMCHD1, SMN1, SMN2, SNX27, SPINK1, SRB1, SRY, ST7, ST7L, STAT3, TFE3, TFEB, TGFB3, THPO, TP63, TP73, UCP2, USP9Y/SP3, UTRN, or VEGFA.

27. The method of claim 26, wherein the RNA transcript is encoded by an isoform of a uORF-containing gene, such as BDNF v11.

28. The method of any one of claims 1 to 25, wherein the RNA transcript is encoded by a gene with a mutation or SNP that creates one or more uORFs, such as: ATP7B, ATRX, BLM, BRCA1, CA2, CCBE1, CD3D, CD4, CDKN2A, CFL2, CFTR, CSPP1, CTNS, DBT, DCAF17, DCLREIC, DFNB31, DLG4, DMD, DNASE1, ETHE1, GALNS, GCH1, HAMP, HBB, HMBS, HR, IGHMBP2, IRF6, ITGAZ, ITGB2, KCNJ11, KCNQ3, LDLR, LRP5, LRP5L, MECP2, MLH1, MSH6, MUTYH, NR5A1, PALB2, PANK2, PEX7, PHYH, PIK3R5, POMC, POMT1, ROR2, SCN2A, SGCA, SGCD, SLC16A1, SLC19A3, SLC2A2, SLC7A9, SPINK1, SRY, STIL, TK2, TMPRSS3, TP53, TPI1, TPM3, TRMU, TSEN54, or ZEB1.

29. The method of any one of claims 1 to 25, wherein the RNA transcript is a non-coding transcript, such as long non-coding RNA (lncRNA), long intervening non-coding RNA (lincRNA), or macroRNA.

30. The method of any one of the preceding claims, wherein the method comprises delivering the compound to a cell by a vector, such as a virus vector (e.g. AAV, lentivirus).

31. A method of modulating the expression or activity of a gene, comprising modulating the presence of a regulatory element in the RNA transcript encoded by the gene according to the method of any one of the preceding claims.

32. A method of increasing, decreasing or restoring protein expression, comprising modulating the presence of a regulatory element in a RNA transcript according to the method of any one of claims 1 to 31.

33. An oligonucleotide targeted to a splicing signal in a RNA transcript for inducing alternative splicing, such that one or more exons comprising a regulatory element are skipped and/or retained.

34. The oligonucleotide of claim 33, as defined in claims 13 to 24.

35. The oligonucleotide of claim 33 or 34, wherein:
(a) the regulatory element is as defined in any one of claims 2 to 7;
(b) the oligonucleotide is for inducing the skipping and/or inclusion of one or more exons as defined in claim 8 or 9;
(c) the oligonucleotide is targeted to a splicing signal as defined in claim 11;
(d) the oligonucleotide is targeted to a target site as defined in claim 12; and/or
(e) the RNA transcript is as defined in any one of claims 26 to 29.

36. The oligonucleotide of any one of claims 33 to 35, wherein the oligonucleotide is conjugated to one or more further compounds, such as a nucleic acid molecule, a peptide, or other chemicals.

37. A conjugated oligonucleotide comprising two or more oligonucleotide of any one of claims 33 to 36.

38. A polynucleotide or a vector encoding the oligonucleotide of any one of claims 33 to 36 or the conjugated oligonucleotide of claim 37, optionally wherein the vector is AAV or lentivirus.

39. A delivery vehicle comprising the oligonucleotide or conjugated oligonucleotide of any one of claims 33 to 37.

40. A modified RNA transcript comprising the absence or inclusion of one or more exons comprising a regulatory element compared to the unmodified RNA transcript.

41. A composition comprising two or more oligonucleotides according to any one of claims 33 to 37, optionally wherein the oligonucleotides are conjugated.

42. A pharmaceutical composition comprising the oligonucleotide according to any one of claims 33 to 36, the conjugated oligonucleotide of claim 37, the polynucleotide or vector of claim 38, the delivery vehicle of claim 39, or the composition of claim 41, and a pharmaceutically acceptable carrier.

43. The oligonucleotide of any one of claims 33 to 36, the conjugated oligonucleotide of claim 37, the polynucleotide or vector of claim 38, the delivery vehicle of claim 39, the composition of claim 41 or the pharmaceutical composition of claim 42, for use in a method of therapy practised on the human or animal body.

44. The oligonucleotide of any one of claims 33 to 37, the polynucleotide or vector of claim 38, the delivery vehicle of claim 39, the composition of claim 41 or the pharmaceutical composition of claim 42, for use in a method of treating or preventing a disease or condition in a subject by modulating the expression of a gene, comprising administering to the subject a therapeutically effective amount of the oligonucleotide, the polynucleotide or vector, the delivery vehicle, the composition or the pharmaceutical composition.

45. The oligonucleotide, polynucleotide, vector, delivery vehicle, composition or pharmaceutical composition for use according to claim 43 or claim 44, comprising modulating the presence of a regulatory element in a RNA transcript according to the method of any one of claims 1 to 30; modulating the expression or activity of a gene according to the method of claim 31; or increasing, decreasing or restoring protein expression according to the method of claim 32.

46. Use of the oligonucleotide of any one of claims 33 to 37, the polynucleotide or vector of claim 38, the delivery vehicle of claim 39, the composition of claim 41 or the pharmaceutical composition of claim 42 in the manufacture of a medicament for the treatment or prevention of a disease or condition in a subject by modulating the expression or activity of a gene.

47. A method of treating or preventing a disease or condition in a subject by modulating the expression or activity of a gene, comprising administering to the subject a therapeutically effective amount of the oligonucleotide of any one of claims 33 to 36, the conjugated oligonucleotide of claim 37, the polynucleotide or vector of claim 38, the delivery vehicle of claim 39, the composition of claim 41 or the pharmaceutical composition of claim 42.

48. The method of claim 46, modulating the presence of a regulatory element in a RNA transcript according to the method of any one of claims 1 to 30; modulating the expression or activity of a gene according to the method of claim 31; or increasing, decreasing or restoring protein expression according to the method of claim 32.


SEQUENCE LISTING (50 sequences)

SEQ ID NO: 1 - DNA, 25 bp, synthetic construct
  source 1..25: mol_type = other DNA
tgggtgggacgggctgaggttcatg

SEQ ID NO: 2 - DNA, 22 bp, synthetic construct
  source 1..22: mol_type = other DNA
ggcaagctggcagacagcaggg

SEQ ID NO: 3 - DNA, 21 bp, synthetic construct
  source 1..21: mol_type = other DNA
attcatgggcctggagatcca

SEQ ID NO: 4 - DNA, 20 bp, synthetic construct
  source 1..20: mol_type = other DNA
ttcccgggccaccaggactg

SEQ ID NO: 5 - DNA, 20 bp, synthetic construct
  source 1..20: mol_type = other DNA
ggggtacttaccgctgatgc

SEQ ID NO: 6 - DNA, 23 bp, synthetic construct
  source 1..23: mol_type = other DNA
taaaatcagcgaaagtgtctgat

SEQ ID NO: 7 - DNA, 22 bp, synthetic construct
  source 1..22: mol_type = other DNA
ttgcttgggcagatgctataaa

SEQ ID NO: 8 - DNA, 25 bp, synthetic construct
  source 1..25: mol_type = other DNA
tttggttgccatttgcttttaactc

SEQ ID NO: 9 - DNA, 23 bp, synthetic construct
  source 1..23: mol_type = other DNA
caaacacctcctctttgttgctt

SEQ ID NO: 10 - DNA, 25 bp, synthetic construct
  source 1..25: mol_type = other DNA
tggccacgttatttaccttttctct

SEQ ID NO: 11 - DNA, 22 bp, synthetic construct
  source 1..22: mol_type = other DNA
ctcctagttagagtctgataac

SEQ ID NO: 12 - DNA, 20 bp, synthetic construct
  source 1..20: mol_type = other DNA
ggaaagcagcggaggggcgg

SEQ ID NO: 13 - DNA, 20 bp, synthetic construct
  source 1..20: mol_type = other DNA
cggcaggagcggctgcgtag

SEQ ID NO: 14 - DNA, 20 bp, synthetic construct
  source 1..20: mol_type = other DNA
ggcaaagctccagcgaccac

SEQ ID NO: 15 - DNA, 22 bp, synthetic construct
  source 1..22: mol_type = other DNA
gtctactcaccagtgacagact

SEQ ID NO: 16-26 - skipped (11 entries)

SEQ ID NO: 27 - DNA, 20 bp, synthetic construct
  source 1..20: mol_type = other DNA
tctccgccgaggccggcgag

SEQ ID NO: 28 - DNA, 20 bp, synthetic construct
  source 1..20: mol_type = other DNA
gaggaaacggctagagaaac

SEQ ID NO: 29 - DNA, 42 bp, Homo sapiens
  source 1..42: mol_type = other DNA
agaatcatgttaagctcggctactgcggagagcccaaggtag

SEQ ID NO: 30 - DNA, 42 bp, synthetic construct
  source 1..42: mol_type = other DNA
agaatcttgttaagctcggctactgcggagagcccaaggtag

SEQ ID NO: 31 - DNA, 42 bp, synthetic construct
  source 1..42: mol_type = other DNA
gccaccatgttaagctcggctactgcggagagcccaaggtag

SEQ ID NO: 32 - DNA, 75 bp, synthetic construct
  source 1..75: mol_type = other DNA
agaatcatgttaagctcggctactgcggagagcccaaggatgttaagctcggctactgcggagagcccaaggtag

SEQ ID NO: 33 - DNA, 75 bp, synthetic construct
  source 1..75: mol_type = other DNA
agaatcatgttaagctcggctactgcggagagcccaaggctgttaagctcggctactgcggagagcccaaggtag

SEQ ID NO: 34 - DNA, 12 bp, synthetic construct
  source 1..12: mol_type = other DNA
agaatcatgtag

SEQ ID NO: 35 - DNA, 15 bp, synthetic construct
  source 1..15: mol_type = other DNA
agaatcatgttatag

SEQ ID NO: 36 - DNA, 18 bp, synthetic construct
  source 1..18: mol_type = other DNA
agaatcatgttaagctag

SEQ ID NO: 37 - DNA, 24 bp, synthetic construct
  source 1..24: mol_type = other DNA
agaatcatgttaagctcggcttag

SEQ ID NO: 38 - DNA, 30 bp, synthetic construct
  source 1..30: mol_type = other DNA
agaatcatgttaagctcggctactgcgtag

SEQ ID NO: 39 - DNA, 36 bp, synthetic construct
  source 1..36: mol_type = other DNA
agaatcatgttaagctcggctactgcggagagctag

SEQ ID NO: 40 - DNA, 75 bp, synthetic construct
  source 1..75: mol_type = other DNA
agaatcatgttaagctcggctactgcggagagcccaagggtgagcggctggcggctgttcaagaagattagctag

SEQ ID NO: 41 - DNA, 66 bp, synthetic construct
  source 1..66: mol_type = other DNA
agaatcatgttaagctcggctactgcggagagcccaagggactacaaagacgatgacgacaagtag

SEQ ID NO: 42 - protein, 11 aa, synthetic construct
MLSSATAESPR

SEQ ID NO: 43 - protein, 11 aa, synthetic construct
MLSSATAESPR

SEQ ID NO: 44 - protein, 22 aa, synthetic construct
MLSSATAESPRMLSSATAESPR

SEQ ID NO: 45 - protein, 22 aa, synthetic construct
MLSSATAESPRALSSATAESPR

SEQ ID NO: 46 - protein, 5 aa, synthetic construct
MLSSA

SEQ ID NO: 47 - protein, 7 aa, synthetic construct
MLSSATA

SEQ ID NO: 48 - protein, 9 aa, synthetic construct
MLSSATAES

SEQ ID NO: 49 - protein, 22 aa, synthetic construct
MLSSATAESPRVSGWRLFKKIS

SEQ ID NO: 50 - protein, 19 aa, synthetic construct
MLSSATAESPRDYKDDDDK